SEQUENCE LISTING (20 sequences)

SEQ ID NO: 1 - DNA, 846 bp, Nitrospira inopinata
atgtttagaacggatgaaataatcaaagccgccaagttgcctccagagggagtggcgatgtcgcggcacattgattacatttactttattcctattttgttcgtgaccatcatcggaacttttcacatgcacacggctttgttgtgcggtgactgggatttctggttggattggaaggatcggcagtggtggccgattgtgactcccatcacaacaattaccttctgcgcagcccttcaatactataactgggtcaattatcgtcagccgtttggggcaacgataaccattttagcgttaggtgccggaaaatgggttgcggtttacacctcttggtggtggtggtccaactatccgccaaatttcgtcatgccggccacgttgcttcctagcgccttggttcttgatttcaccttgttgctaactagaaactggactttgaccgcagtgatcggggcctggatgtacgcgattttgttctatccgagcaattggcctatctttgcttacagccatactccgcttgtggtggatgggaccttgctttcatgggccgactatatgggctttatgtatgtgcggaccggaactcctgaatatatccgtatgattgaagttgggtcgctgcggacgttcggtgggcacagcacgatgatttcctcgttctttgctgcattcgcctcttcattgatgtacatcctgtggtggcagtttggaaagtttttctgcacgtcctatttctacttcacggatgacaagaagcgaacgaccaaagtttacgatgtctttgcctatgcaacattggctcacgcggataaggccaaactctctggggggaaagcatga

SEQ ID NO: 2 - protein, 281 aa, Nitrospira inopinata
MFRTDEIIKAAKLPPEGVAMSRHIDYIYFIPILFVTIIGTFHMHTALLCGDWDFWLDWKDRQWWPIVTPITTITFCAALQYYNWVNYRQPFGATITILALGAGKWVAVYTSWWWWSNYPPNFVMPATLLPSALVLDFTLLLTRNWTLTAVIGAWMYAILFYPSNWPIFAYSHTPLVVDGTLLSWADYMGFMYVRTGTPEYIRMIEVGSLRTFGGHSTMISSFFAAFASSLMYILWWQFGKFFCTSYFYFTDDKKRTTKVYDVFAYATLAHADKAKLSGGKA

SEQ ID NO: 3 - DNA, 1260 bp, Nitrospira inopinata
atgaacgtcaaacacgtcttcaagctgtggatgctgggattctgcggagtggcgacgttggcgttcacgccggtgtttgatgctgctccagttcttgctcacggggagcgttcgcaagaaccgtttctgcggatgcgcaccgtgaattggtatgacactgaatgggtggggaaaagcactgcggtaaatgatgttacatacatgaggggcaagtttcatctgtctgaagactggcctcgtgcggtagtgaaaccccatcgaacgttcgtcaatgtcggctctcctagctccgtctttgtgcggttaagcacgaaggttggtggggtgccgatgtttgtgtctggtcctatggaaatcgggcgtgattatgaatatgagatcacgttgaaggcgagacttcctggacatcatcacattcaccctatgttttctgttaaagaggctggtcccattgccggaccgggtgggtggatggatatcacgggccgatacgctgattttacaaacccgatcaagactctgacgggggaaacatttgactcggaaacagagggtgggatgaccggaattatgtggcatatattctgggcatctgttgccttgttctgggtgggttggttcatggttcgcccgatgtacttgattcgggctcgtgtgcttgcggcttatggtgatgaacttctgttggatccggttgatcgcaagctcgcaataggtcttctcgtatttacggtggcggttgtcactatcggttatctcgctgcggaggcgaagcatcctattaccgtgcccctgcaggctggtgaagcaaaggttaaaccgcttcctataaaaccgaatccattggtggttgaagtcacccacgccgaatatgacgtgccgggtcgtgctcttcgtatgacggttcacgccactaacaatgggactgagcctgtcagtatcggtgaattcacaacggctggtattcgatttacaaataaagtaggagcagcgaagctcgatccgaactatccacaggagcttattgctacagccggactgaccatggataatgaggctccgatacagccgggtcagactgttgacattcacatagaatcaaaggatgttctatgggaggttcagcggctggttgacattcttcacgatccggatcagcggtttgctgggttgttgatgtcatggactgaatcgggagaacgtcttattaaccccgtgtgggctcctgtgcttcctgtctttacacgattgggagcataa

SEQ ID NO: 4 - protein, 419 aa, Nitrospira inopinata
MNVKHVFKLWMLGFCGVATLAFTPVFDAAPVLAHGERSQEPFLRMRTVNWYDTEWVGKSTAVNDVTYMRGKFHLSEDWPRAVVKPHRTFVNVGSPSSVFVRLSTKVGGVPMFVSGPMEIGRDYEYEITLKARLPGHHHIHPMFSVKEAGPIAGPGGWMDITGRYADFTNPIKTLTGETFDSETEGGMTGIMWHIFWASVALFWVGWFMVRPMYLIRARVLAAYGDELLLDPVDRKLAIGLLVFTVAVVTIGYLAAEAKHPITVPLQAGEAKVKPLPIKPNPLVVEVTHAEYDVPGRALRMTVHATNNGTEPVSIGEFTTAGIRFTNKVGAAKLDPNYPQELIATAGLTMDNEAPIQPGQTVDIHIESKDVLWEVQRLVDILHDPDQRFAGLLMSWTESGERLINPVWAPVLPVFTRLGA

SEQ ID NO: 5 - DNA, 810 bp, Nitrospira inopinata
ctagtagcctgctttggcgttggggttgacctggctggggaacggatccaggatgctcttgggcgccccgttccaaatcacatccgccaagttcgacatgcggctcacaatctgcgccgccacgccgccggccgccccgaacaacccgcaccagcccaacgtcacaaagccccaatgcaacggcgccgcaaacaactcgtccacaaaccaaaacgcatggccccactcattgagccccacgttcggcaaaatgaacatcggccccaccaccgccgccaccaacggaaacgacgtcgcctggctatacaacggcaaccgcgtctgcgcatacagataactcgagaccccgcacgtaatgtacaacgggaacgtcccgtaaaacgccacaatgtgactggccgtaaaactcgtgtcccgaatgatcacctgatgccacgccgcatcctgctccaacgtgtagctgcccgcataatagaccccccacacgtagcaggccaaccaccccatccagtaaaaataccgcttcaactccgtcttcggatccaaattcgccaaattccgatcccgcgtcacccaaatccaccccaccgaaatcgcaaaaaataacgcattggccacaatgttaaaccgccataaccccatccacaccgcgtcaaactccggcgtcatcgagtccaacccgtgcgaatacccaaacgtccgctgatacaacacccaaaaaatcccaatcgccaacatcgcaaaccacccaatcttccacggccgcgaatcataccactgcgaaatgtcatacccccgctctgccgccat

SEQ ID NO: 6 - protein, 269 aa, Nitrospira inopinata
MAAERGYDISQWYDSRPWKIGWFAMLAIGIFWVLYQRTFGYSHGLDSMTPEFDAVWMGLWRFNIVANALFFAISVGWIWVTRDRNLANLDPKTELKRYFYWMGWLACYVWGVYYAGSYTLEQDAAWHQVIIRDTSFTASHIVAFYGTFPLYITCGVSSYLYAQTRLPLYSQATSFPLVAAVVGPMFILPNVGLNEWGHAFWFVDELFAAPLHWGFVTLGWCGLFGAAGGVAAQIVSRMSNLADVIWNGAPKSILDPFPSQVNPNAKAGY

SEQ ID NO: 7 - DNA, 1710 bp, Nitrospira inopinata
tcaacgatctttcctctctcgtcgacgccagccagcaattgccaacgtaccggccagcatcatacctccgcccaaccccccgatcgacagcttcccgcccgggccatcgagatcaagcagagagtgcttgcgctcgctctcaagcttatcgacacgggccaacaacttttgtgtttccttaagccgagtgtcatcgtccatgatttccacataggcccgattcatcgcagcccaaccaaccgtataggtatatccccaatactggtgagccaagctgacgtgcaattgaaccaggtgatcctccgccatttcgaacagcttgagctcgttggccgccgggttatttccctttgaccagtaaatctggaagaactgctcaaagccgtccttcaccggaggaggcggtgcgggccggttggttttttgccccgtcaacaacccggccttgtactgctcttcgactacgtgatgagcctcgtcgtacttatcaagaccggagtacgtacctttatccatgaactccatccaggcgcgggcgtaactttccgagtgacagttcgtacaggttttaacccacgcatcgagccgcttttccgaccaatcggtcgagatattctctcggatgccaggaacgaacggatagttggcccatcgaaccttgcgcaccacgttgtgggttatcttaccttgatattccatgtggcagaattggcatgtcggggcactcaatccacccttggcaatggcttctttaatgggaatattgaaattccacttatctttgtctctctgatacttcaatccatgcttggagagggagtaggcctcccagttgttgtgatcggccccactgtgacactgagcgcaattttccggcttgcgcgattccgcgactgagaattcatgacgcgagtgacaagtatcgcacttgttctgattgacgtgacaaccggtacagccatcggcgatttctctttgaggcatcccggcatagacgtccacttccacgttagccttataatccaaggcatgcgaaggacgccccttgggccactgatcttttggccatatgatggtgtcacgctccgattctcgttcagcgaattcctgaagatggcacgtaccgcaggtgttcgccgtcgccagcttaatgtccttgcgatgatcggccttccctttggcattgatttcaaagtgacaatcaatgcacccaacttcttttagctgctcccccttgcccaacttgcccatcgaccgaaggttttcctcgatttgctcaagcttcgccttcttgtaataggtctcatccttgggcgtcagtttacggatcttatccagattggcatgcgtgctgcgcttccacgcagccacccaccccggcgattcatccgtatgacatttgacgcactgctcacggcttgcgacttccttgactgcctgcgggggcttatagaacgtcgtgggatcgaaatacttactgaaagtgacgggctcccagtactgaccgtaaattccctttccggccccttgctcaggatcgaggtaccttttcaccagagcctcgtacagctcctttggagaagccgaacggtcgatcttcagtgcctcataagtttccttcggcaccgtcgggaagtccgcttgcgccggtgcggccagtaaaacgccgcagacgagcatcacaaacttctgcgcaaacttgctgctcat

SEQ ID NO: 8 - protein, 567 aa, Nitrospira inopinata
MSSKFAQKFVMLVCGVLLAAPAQADFPTVPKETYEALKIDRSASPKELYEALVKRYLDPEQGAGKGIYGQYWEPVTFSKYFDPTTFYKPPQAVKEVASREQCVKCHTDESPGWVAAWKRSTHANLDKIRKLTPKDETYYKKAKLEQIEENLRSMGKLGKGEQLKEVGCIDCHFEINAKGKADHRKDIKLATANTCGTCHLQEFAERESERDTIIWPKDQWPKGRPSHALDYKANVEVDVYAGMPQREIADGCTGCHVNQNKCDTCHSRHEFSVAESRKPENCAQCHSGADHNNWEAYSLSKHGLKYQRDKDKWNFNIPIKEAIAKGGLSAPTCQFCHMEYQGKITHNVVRKVRWANYPFVPGIRENISTDWSEKRLDAWVKTCTNCHSESYARAWMEFMDKGTYSGLDKYDEAHHVVEEQYKAGLLTGQKTNRPAPPPPVKDGFEQFFQIYWSKGNNPAANELKLFEMAEDHLVQLHVSLAHQYWGYTYTVGWAAMNRAYVEIMDDDTRLKETQKLLARVDKLESERKHSLLDLDGPGGKLSIGGLGGGMMLAGTLAIAGWRRRERK

SEQ ID NO: 9 - DNA, 969 bp, Nitrospira inopinata
tcagttttgccgttgaatctgctcagcagaagggattttgtatatccaatatcccccttctttatgtattaattgcaacgcatcaagatccatcggtctaaagttggaaaacggcaacattttcgccaacagtatgttgctggctccgctttctcggaggaaataagcacgcacttgcttctcggagagagactgaagggtatagcttgtgtagtcgttgtttgtcagccaggctttcacctgcccgctgagtccatggacgtttcctgtcaagggaaaatccttaaatgcaacatctatgcgatcaggtcgcatcagtcccaacttataaatgtcgctcacatgcacgaccacgtatgcttctcgcgaaccaaccagctcgcgcaacattgcggcaccttgatgagggtacgccatgagagcttcaataaatcggttaaactgcgcgcgttcttccggagaagcctgcttcccccaaaacttctgctcgtactgttcaatcgcttcaaatcggtctctccagtacgacggggcgatgaccggcaagccaagataagcggtgaagagcgtcggacgatccgacaaaagggccagttttctcgaagtatcccaccatgcgacgatgacggcatcgttcggcacatgtttggcgatggcggacgatacggcgatcgtttctgaaagcccttcttccatggataaaatcggttctggaaacttattttccacatacagaagaacaggatcgaaaccggagcggtttgcgcgatagacaaaggcgagcggttgatcgattgcgtccacttgtacttcgagcttaccgattgtcagatatggataattttccaatcccaggttagggaatttatccgggccgccctcctccaccacacgatagtgatagggcggtggttccggttgaaaccacaaatagacaaaccatcctactaaaaaaaggcctcccgccac

SEQ ID NO: 10 - protein, 322 aa, Nitrospira inopinata
MAGGLFLVGWFVYLWFQPEPPPYHYRVVEEGGPDKFPNLGLENYPYLTIGKLEVQVDAIDQPLAFVYRANRSGFDPVLLYVENKFPEPILSMEEGLSETIAVSSAIAKHVPNDAVIVAWWDTSRKLALLSDRPTLFTAYLGLPVIAPSYWRDRFEAIEQYEQKFWGKQASPEERAQFNRFIEALMAYPHQGAAMLRELVGSREAYVVVHVSDIYKLGLMRPDRIDVAFKDFPLTGNVHGLSGQVKAWLTNNDYTSYTLQSLSEKQVRAYFLRESGASNILLAKMLPFSNFRPMDLDALQLIHKEGGYWIYKIPSAEQIQRQN

SEQ ID NO: 11 - DNA, 3438 bp, Nitrospira inopinata
ttatactttaatcttgatgtgctcacctttcagccacttgatcataaattcattttcctggcccggcgtgaaccctgtccgcaccggttcccacgggccacgtgccccaatgccgccatcctccgccttcgtgatacggataaggcattctttcgggacggtgttgatggcatggtgatcgacttgatagccccacttgaacttccaggcaatggcatgtttgcctggcaatgagtcggtttgatgcatcggcatcagccagttccgcgtaaacgattgctgacacccatatctaaagtttgactgatagccggtatcgatggcaatcgcacgtccatcaggtcttgtttcgtgcccttttacggactttggcgtcgccacgaacggagcgtgcttggccatcgtcacgtggtagggataggcggggttgtacttggcccgaatcatcagccgagcgaccttgtaataggggtcactgggcttccaaccacggtaaggccgatccaccggatttccatcgacataaacgtagtcgccatcgttgatcccacgatccttcgcagcctggggattgatgtgaagctgatgttctcccacgcccggagtccgtttatccatacgatagggatcgccgaagttcgactcatagatctgcacccagtcgttcaccgaccactggctgtgaacacggtgacgagtctttggcgtaacacagtagaactggtaccccttctcccacagcggattactgtgccgcttgatctcatcccacgagagcttaatgttacggacggttttgtcatcatggtgctgagccgtgatgggtatcccgtagtcatcaggccgcacatagggattggttgtaaagatggcattgggcaggtacggagttgcctctggaccctcccgatgcgaaatgaaattttctccatactcgatggcttcggcttccggtcgatagttttcatatcttccgcttcgcgtccacatgggtttggactcgttggtctcttcccagaatgggtggcgcggataggttctcaccatgaccatccaccccttttcagacttgagcatgacgtccgccgagtagccgtagaacgtgcttgaggcgtcaagcattcgctgcgcatagacatcaacacgattggcatagaccatcgcgaagtaatctttcatccgtttatcgccggtcatgtcggacagtttggctgccactccggcaaacgtatcgaggtcgttacgggtgtcatagaggggcctgattccgcccttccagatctgaacccatgggttggataccgtgatggtcatttccggatacgtaaattccatccaagagttgcaagcaaacgcgatatccgcatggttgatgtctgatgtcatttcgatgtcttgagtgatcagacattcaatgttcggatccacgtttttgaccatgtcatagtggtgcttggcgttattgaccacgttgacgtttgtcacccagcggaatttactcggagtcggcatgtgcgtctttccggtaaacaccttgcgtccatacttaggtgtattgacgatcaaagccgtgtcaccgtgattccagtacccgacttcttccccgtaataataggacctcgtatggatctcctttccgtgcgcattgggatccaacgtgatattgaacggatcttctcccgtgtgaacactgagtccggcccccgaccatggcgtggcagtccatgcgccggccttatagtttccggcccaagtatgctgaccggtcccgaacttgccaacgttccccgtgataatcagcaccatcgcagccccacgggcgttgatggtctgatggaagtagtggcacgtgccttcgccattgtgaatcgcggccggtttaatagtgcccgaatcacgagcccatcgcacaataaggtctttcggcgttcgcgtgatctggtgaaccgtatcaagatcataatcctggaaatgcaccatgtacatttgccatataggcatggcatcaatttcacgcccgttcagcaatttgactctgtacgtaccggtcagggccgcatcgataccgctgttcacatagtgccatccgacctgctctcgatggaggggaacagcctgctttttattgaggtcccacaccatcatcccgcctaaacgctgaatctgctcgggcttgagcgactgaattctccccgaatagctttttgaaaaatcaggaaatttgtaatcaggaatgacatcgcgcgggtccaaatattggagcgtgtccgttcgcacaagaattggggcatcggtaaaggatttcagaaaatcaacgtcgtgcatgttctcatcgacgataatcttcatcgcccccaaaaagagcgcgccatcagactgcggacgaagcggcatccagtaatcagcccgataggcagtggggttgtattcgggagtgatcacaacaaccctcgcgcctcgctcgatacattcgagcttccaatgggcctccggcatcttgttttcaacaaagttctttccccagctcgtgttcaatttagaaaaacgcatgtcggataggtcaacgtcagacccctggacaccggaccaccagggatgggcaggattttgatccccgtgccaagtatagttggaccaataacggcctccctgcgcctggtccggccccacctttctaatccaagtatccagaagagcgttgatcccgccgttcatcctggtgttgcccatttttccaatgatcccaagcaccggcatcccagctcggtgcttgaaacagcgggtaccggcccccttcatcatctcgatcatttcaggcgcatatccctgctcccggagacgcctggccccagcctcgccactataccgcgtggcgataatgatcatggccttggccgcataagtgaacgccgtatcccaagacactcgaagcatgtcatccaggaaccggctatcgaatttatacttgcgcttggtttccggcgtcagttcgggagcaccatcatccatccactgcttccatccctttcgcatcaagggccccttcaaccgatacggcccgtagacacgccgatggaacgtaaaccccttcaggcacatacgaggattgtgcgcgaacgtcccccgatttccataaagatcttcataggtctggtggtcataattttgctcaacgcgcatgaccacgccgtttctaacaaatgcccgcacccggcaggcatgcgtgtcattgggcgagcaaacccatgtaaatgatgaatcgtatcggtactgatcatgatagacacgctcccaggaccgatctggatactcgccgagcgggtttccaacctcaataaccggttgcagcgcggttaacgccaagactttatcggcaaccgccacagcggcaaccgtccccacggataccttcaaaaactgcctacgcgacaagaacat

SEQ ID NO: 12 - protein, 1145 aa, Nitrospira inopinata
MFLSRRQFLKVSVGTVAAVAVADKVLALTALQPVIEVGNPLGEYPDRSWERVYHDQYRYDSSFTWVCSPNDTHACRVRAFVRNGVVMRVEQNYDHQTYEDLYGNRGTFAHNPRMCLKGFTFHRRVYGPYRLKGPLMRKGWKQWMDDGAPELTPETKRKYKFDSRFLDDMLRVSWDTAFTYAAKAMIIIATRYSGEAGARRLREQGYAPEMIEMMKGAGTRCFKHRAGMPVLGIIGKMGNTRMNGGINALLDTWIRKVGPDQAQGGRYWSNYTWHGDQNPAHPWWSGVQGSDVDLSDMRFSKLNTSWGKNFVENKMPEAHWKLECIERGARVVVITPEYNPTAYRADYWMPLRPQSDGALFLGAMKIIVDENMHDVDFLKSFTDAPILVRTDTLQYLDPRDVIPDYKFPDFSKSYSGRIQSLKPEQIQRLGGMMVWDLNKKQAVPLHREQVGWHYVNSGIDAALTGTYRVKLLNGREIDAMPIWQMYMVHFQDYDLDTVHQITRTPKDLIVRWARDSGTIKPAAIHNGEGTCHYFHQTINARGAAMVLIITGNVGKFGTGQHTWAGNYKAGAWTATPWSGAGLSVHTGEDPFNITLDPNAHGKEIHTRSYYYGEEVGYWNHGDTALIVNTPKYGRKVFTGKTHMPTPSKFRWVTNVNVVNNAKHHYDMVKNVDPNIECLITQDIEMTSDINHADIAFACNSWMEFTYPEMTITVSNPWVQIWKGGIRPLYDTRNDLDTFAGVAAKLSDMTGDKRMKDYFAMVYANRVDVYAQRMLDASSTFYGYSADVMLKSEKGWMVMVRTYPRHPFWEETNESKPMWTRSGRYENYRPEAEAIEYGENFISHREGPEATPYLPNAIFTTNPYVRPDDYGIPITAQHHDDKTVRNIKLSWDEIKRHSNPLWEKGYQFYCVTPKTRHRVHSQWSVNDWVQIYESNFGDPYRMDKRTPGVGEHQLHINPQAAKDRGINDGDYVYVDGNPVDRPYRGWKPSDPYYKVARLMIRAKYNPAYPYHVTMAKHAPFVATPKSVKGHETRPDGRAIAIDTGYQSNFRYGCQQSFTRNWLMPMHQTDSLPGKHAIAWKFKWGYQVDHHAINTVPKECLIRITKAEDGGIGARGPWEPVRTGFTPGQENEFMIKWLKGEHIKIKV

SEQ ID NO: 13 - DNA, 1290 bp, Nitrospira inopinata
ttacaaccaggtcactcgctctgccggtctgatataaatcggttcttcgacttgaatacgggccacctctttgcctgacttgttaaaaccaagaaccgtgtcgttgtacatctcaaaccgcttgccatgaatttgggtctcaaagacttttggcccaggaatcacgtcatagcggaagatgatctgttgactggctcgccacaactgaaggacggccagcaattcccggcttgggacgagatatttttcgattgcgttgtctacgccaggaccgaacatctgtctcgcatagcctcgcgggctatgccgtggaggaatgtagaagccgttcggttccgtcccccattgcgggtagaggggtaaggccacttgttcgacacggatggcgtaatacagcggatgccaccgatcctcagcccacagaccgtcttctccgatacgaactaagctctgcattctgatctttcctacgcaggctgccatacaccgcgtttccattggttctccgccggtaagaggatcttttccttcgatgcgcggataacaggcaatacacttttctgagaccctggtggtgcctcgatacatgggctttttgtatgggcactgttcaacgcattttttgtatcctcgacatcgattctgatcgatgagaacaattccatcttctggccgtttgtagatcgcttttcttggacaagcggctaggcagccagggtaggtgcaatggttacagatacgttggaggtaaaagaaaaacgtttcatgctccggcaggctgctgccggtcattttccagggctcatccttcgagaaacctgtcttgtcgatcccctcgaccagcgcccgcatcgacgttgccgtatcttcgtagatattgacaaaccgccactcctggtctgttgggatgtagccgattgcagcctgccccactttggcccctgcatcgaaaatggtcatcccttcgaagaccccgtagggtgcatggtgtttccgtcctactcgaacgttccacacctggccgccgggattgacctgctcgataagctgagtgattttgacatcgtaaaattgcgggtacccgccatagggcttcgtctcgacattgttccaccacatgtactcctgacctttcgagaaaagccaggttgacttatccgccatggaacacgtctgacaggccagacatcgattgatgttaaacacaaaggcaaattgccatttgggatgccgctcctcatagggataaagcatctttcgtcctaactgccagttataaacttcaggcat

SEQ ID NO: 14 - protein, 429 aa, Nitrospira inopinata
MPEVYNWQLGRKMLYPYEERHPKWQFAFVFNINRCLACQTCSMADKSTWLFSKGQEYMWWNNVETKPYGGYPQFYDVKITQLIEQVNPGGQVWNVRVGRKHHAPYGVFEGMTIFDAGAKVGQAAIGYIPTDQEWRFVNIYEDTATSMRALVEGIDKTGFSKDEPWKMTGSSLPEHETFFFYLQRICNHCTYPGCLAACPRKAIYKRPEDGIVLIDQNRCRGYKKCVEQCPYKKPMYRGTTRVSEKCIACYPRIEGKDPLTGGEPMETRCMAACVGKIRMQSLVRIGEDGLWAEDRWHPLYYAIRVEQVALPLYPQWGTEPNGFYIPPRHSPRGYARQMFGPGVDNAIEKYLVPSRELLAVLQLWRASQQIIFRYDVIPGPKVFETQIHGKRFEMYNDTVLGFNKSGKEVARIQVEEPIYIRPAERVTWL

SEQ ID NO: 15 - DNA, 1308 bp, Crenothrix polyspora
atgaaaacactatggcaaaataatccgtgtgcaacaatggccaaaaccatcagttaccggaatgctaacgcactaaagcagccctttacgaaaggcttgttattcctgggtacgctactttcggtgtatatgttgaccctacagcctgtcatggcgcacggggaaaagaacctggaaccctatgtcagaatgcgtaccgtccaatggtatgacgtgcaatggtccaagcagaaatttaatgtcaacgatgaaattagcgtaaccggtaaatttcatgtggccgaagattggccgatcagcgtacccaagccggatgcggcgttcttaaatatctcaacaccaggccccgtgctgatcagaaccgaacgttacttaaacggcaagccctacatgaattcagtggccttacaaccaggcggcgactatgacttcaaggttgtcctgaaaggacgcttaccaggacgttaccacatccatcctttctttaacctaaaggatgcagggcaagtcatggggccgggcgcatggttggatattgcaggcgatgccagcgattttaccaataacgtccagaccatcaatggcgaactggtcgatatggaaaacttcgggttgggtaacggcatcttctggcacagcttttgggctttgttgggtacggcctggctgctttggtgggtacgccgccccttgtttattgagcgttaccggatgttgcaagcaggcttggaagatgaattggtgactccattggacagaaatattggcaaagcaatagtcatcggcgtgcctgttctggtgtttatgttttataccatgacggtgaacaaatatcccaaggccatacctttacaagcctcactagaccaaatcctgcctctttctgcccaagtcaatgccggcgtagtcgatgtcgaaacggtgcggacagaataccgcgtcccaaaaagatcgatgactgtcagcttaaagatcaaaaatggcagcgacaagccgattcagataggtgaatttgcaacgggcggtgtacgcttccttaaccaagctgtatctgtacctgaccagaacaatgcagaaagtgttatcgcgaaagaaggcttaatattggataatccagcccccatccagccaggtgaacaacgtacagtgttaatgaccgcaagcgatgccttgtgggagtcagaaaaactggacggcctgattaacgatgccgacagccgtattggcggcttgatctttttcttcgacagtgagggtgaacgcactatttccagcatcacctcggctgtcattcctaaatttgattaa

SEQ ID NO: 16 - protein, 435 aa, Crenothrix polyspora
MKTLWQNNPCATMAKTISYRNANALKQPFTKGLLFLGTLLSVYMLTLQPVMAHGEKNLEPYVRMRTVQWYDVQWSKQKFNVNDEISVTGKFHVAEDWPISVPKPDAAFLNISTPGPVLIRTERYLNGKPYMNSVALQPGGDYDFKVVLKGRLPGRYHIHPFFNLKDAGQVMGPGAWLDIAGDASDFTNNVQTINGELVDMENFGLGNGIFWHSFWALLGTAWLLWWVRRPLFIERYRMLQAGLEDELVTPLDRNIGKAIVIGVPVLVFMFYTMTVNKYPKAIPLQASLDQILPLSAQVNAGVVDVETVRTEYRVPKRSMTVSLKIKNGSDKPIQIGEFATGGVRFLNQAVSVPDQNNAESVIAKEGLILDNPAPIQPGEQRTVLMTASDALWESEKLDGLINDADSRIGGLIFFFDSEGERTISSITSAVIPKFD

SEQ ID NO: 17 - DNA, 753 bp, Crenothrix polyspora
atgtcagcaaaactttcaaagccaacgtttaagccgtataccggcgagaaggcgcgtatcacccgcgcttacgactacctgatcctagtattggcgctgttcttgttcatcggttctttccatctgcattttgccctcactgtgggcgactgggatttttgggtagactggaaggacaggcaatggtggccattggtcaccccactcattggcattacctttccggcggcagtacaggccgtactatggagtaacttccgcttgccattgggtgcaaccctgtgtgttgcctgtttgtcgataggtacctggattgcccgtgtctttgcataccactactggaattattttcccatcaacatggtgatgccatcgacactgctgcctagtgcgctggtcttggacggcatcctcatgttaagtaatagcctgacagtgaccgctattttcggcggctctgctttcgccttactgttctaccctgcaaactggcccatcttcggtatgttccatctccccgttgaagcgggcaacagccaattgaccctggccgatatgtttggcttccagtacatccgtaccggtatgccggaatatcttcgtattattgagcgggggacgttacgtacttatggccaaattgccacaccgctgtcggccttttgctcagcgctgttatgcactttgatgtacaccttgtggtggcatatcggcaaatggtttgccacgacccgttatcttaaaagaatctaa

SEQ ID NO: 18 - protein, 250 aa, Crenothrix polyspora
MSAKLSKPTFKPYTGEKARITRAYDYLILVLALFLFIGSFHLHFALTVGDWDFWVDWKDRQWWPLVTPLIGITFPAAVQAVLWSNFRLPLGATLCVACLSIGTWIARVFAYHYWNYFPINMVMPSTLLPSALVLDGILMLSNSLTVTAIFGGSAFALLFYPANWPIFGMFHLPVEAGNSQLTLADMFGFQYIRTGMPEYLRIIERGTLRTYGQIATPLSAFCSALLCTLMYTLWWHIGKWFATTRYLKRI

SEQ ID NO: 19 - DNA, 774 bp, Crenothrix polyspora
atggctacaaccactgaaaaaatcaaggtaataaccgaacaggccaaaatgccaccctggtatttgaaggatttataccgctatctgtcggctttcggcatactgaccgccatctatatgggtttccgtatttatcagggggcgtatggtgtctcaacaggattggattcaaccgcccccgattttgatgtctactggatgcgtctgttcaactttaacgtgacttttgttacgctttttgcaggcgtttcatggggatggttatggtttacccgggataaaaacctggacaagcttgaacctaaggaagaaatccgccgctattttacgttgaccatgttcattagcgtctatacctttgctgtatattgggctggcagttactttgccgagcaagataactcctggcatcaggtcgctattcgagacacaccttttaccgccaaccatatcattgaattttatttcaatttccccatgtacattatccttggcggttgcgcctggctttatgccagaacacggctgccgctttatgccaaaggcatttcactgccgttgacgctggctgttgtcgggccttttatgatattggtgagtgtcggttttaatgaatgggggcataccttctggtttcgtgaggagttttttgctgcgccgatccattacggcttcgtgattggggtttggtttgcgcatggcgtggggggtatattgctgcaaggtgtgacccgtttgattgagttgctagacgcacaggaagacgtggcttaa

SEQ ID NO: 20 - protein, 257 aa, Crenothrix polyspora
MATTTEKIKVITEQAKMPPWYLKDLYRYLSAFGILTAIYMGFRIYQGAYGVSTGLDSTAPDFDVYWMRLFNFNVTFVTLFAGVSWGWLWFTRDKNLDKLEPKEEIRRYFTLTMFISVYTFAVYWAGSYFAEQDNSWHQVAIRDTPFTANHIIEFYFNFPMYIILGGCAWLYARTRLPLYAKGISLPLTLAVVGPFMILVSVGFNEWGHTFWFREEFFAAPIHYGFVIGVWFAHGVGGILLQGVTRLIELLDAQEDVA

(19) United States
(12) Patent Application Publication
(10) Pub. No.: US 2018/0305658 A1
(43) Pub. Date: Oct. 25, 2018

(54) Composition of Bacterial Mixture and Uses Thereof

(71) Applicant: T. Madison Glimp, Denver, CO (US)

(72) Inventor: T. Madison Glimp, Denver, CO (US)

(21) Appl. No.: 15/494,438

(22) Filed: Apr. 21, 2017

(51) Int. Cl. C12N 1/20; C12N 9/06; D06M 16/00; A61L 9/01

(52) U.S. Cl. CPC C12N 1/20; C12N 9/0044; A61L 9/01; D06M 16/003; C12Y 107/01004

(57) ABSTRACT

The disclosure provides bacterial compositions and methods of use thereof for ameliorating malodor in fabrics. More specifically, the invention provides bacterial compositions comprising bacteria capable of complete nitrification.

### FIELD OF THE INVENTION

The present disclosure relates generally to the field of non-pathogenic bacteria. Specifically, the present disclosure relates to compositions of bacteria and methods of using the disclosed compositions to treat fabrics.

### BACKGROUND OF THE INVENTION

Bacteria occur widely in soils and waters, reaching populations sometimes in the million per gram or per milliliter. Most bacteria are not dangerous to humans; conversely, some bacteria provide health benefits. Such health benefits relate to treatment of human skin. Bacteria come in all sorts of shapes and sizes and yet each group has somewhat different preferences for habitat, foods and the level or needs for oxygen.

Nitrification is a two-step process where ammonia is first oxidized to nitrite by ammonia-oxidizing bacteria and/or archaea, and subsequently from nitrite to nitrate by nitrite-oxidizing bacteria. Nitrification can also be carried out by a single organism capable of oxidizing both ammonia and nitrite (see Daims et al., Nature, 2015 (528) 504-509; van Kessel et al., Nature, 2015 (528) 555-559; both of which are incorporated herein in their entirety).

Certain fabrics, including articles of clothing, furniture coverings, carpet, automotive upholstery, and others, are unable to or are not recommended for common day wash-machines. While some fabrics are unable to go through the wash, others have technological aspects that deplete with each wash, yet other fabrics are too large or attached to items that are too difficult logistically to wash. Still, certain articles of clothing are recommended to not be washed by the manufacturer, or are preferred unwashed by the consumer and user, as the articles of clothing contain technology or design aspects requiring special care and handling.

### SUMMARY OF THE DISCLOSURE

It is against the above background that the present invention provides certain advantages and advancements over the prior art.

Although the invention disclosed herein is not limited to specific advantages or functionalities, the invention provides a bacterial composition comprising at least one species of bacteria wherein the at least one species of bacteria is capable of catalyzing complete nitrification.

In some aspects, the bacterial composition comprises at least one species of bacteria selected from Nitrospira, Nitrosomonas, Nitrosococcus, Nitrosospira, Nitrobacter, Nitrospina, Nitrococcus, and combinations of thereof.

In some aspects, the bacterial composition comprises at least two species of nitrifying bacteria.

In some aspects, the bacterial composition is in a form selected from the group consisting of liquid, concentrated, frozen, freeze-dried, and powdered.

In some aspects, the bacterial composition comprises at least one additional species of bacteria which serves metabolic functions ancillary to the nitrifying bacteria.

In some aspects, the bacterial composition comprises at least one species of Nitrospira bacteria.

In some aspects, the bacterial composition comprises at least one urease inhibitor.

In some aspects of the bacterial composition, the bacteria capable of catalyzing complete nitrification is a recombinant host comprising at least one nucleic acid encoding a polypeptide capable of oxidizing ammonia or ammonium to nitrite and at least one nucleic acid encoding a polypeptide capable of oxidizing nitrite to nitrate, wherein at least one nucleic acid encoding a polypeptide capable of oxidizing ammonia or ammonium to nitrite is recombinant; at least one nucleic acid encoding a polypeptide capable of oxidizing nitrite to nitrate is recombinant; or at least one nucleic acid encoding a polypeptide capable of oxidizing ammonia or ammonium to nitrite is recombinant and at least one nucleic acid encoding a polypeptide capable of oxidizing nitrite to nitrate is recombinant.

In some aspects of the bacterial composition, the recombinant host bacteria comprises at least one polypeptide capable of oxidizing ammonia or ammonium to nitrite where such polypeptides are selected from ammonia monooxygenase, hydroxylamine oxidoreductase, hydroxylamine dehydrogenase, methane monooxygenase, and combinations thereof.

In some aspects of the bacterial composition, the recombinant host bacteria comprises at least one polypeptide capable of oxidizing ammonia or ammonium to nitrite where such polypeptide is 90% homologous to SEQ ID NO: 2, 4, 6, 8, 10, 16, 18, 20, or a combination of these sequences.

In some aspects, the bacterial composition comprises at least one polypeptide capable of oxidizing nitrite to nitrate by way of nitrite oxidoreductase.

In some aspects, the bacterial composition comprises at least one polypeptide capable of oxidizing nitrite to nitrate where such polypeptide is 90% homologous to SEQ ID NO: 12, 14, or a combination of these sequences.

Another aspect of the invention is a method of treating a fabric, the method comprising applying to the fabric, an effective amount of the bacterial composition described above.

In some aspects, the method includes applying an effective amount of the bacterial composition requires spraying the bacterial composition on to the fabric at least once.

In some aspects of the method, the fabric is wiped with an applicator dampened with water prior to the application of the effective amount of bacterial composition.

In some aspects of the method, the fabric is an article of clothing.

In some aspects of the method, the fabric is denim.

Some aspects of the invention include a kit useful for the treatment of a fabric, where the kit comprises an effective amount of the bacterial composition described above and instructions for using the bacterial composition.

In some aspects of the kit an applicator used for applying the bacterial composition to a fabric is included.

In some aspects of the kit, the bacterial composition is packaged as a concentrate which can be diluted with water.

### DESCRIPTION OF EMBODIMENTS

Unless otherwise defined, all technical terms used herein have the same meaning as commonly understood by one of ordinary skill in the art to which this disclosure belongs.

The articles “a” and “an” are used herein to refer to one or to more than one (i.e. at least one) of the grammatical object of the article. By way of example, reference to a “nucleic acid” means one or more nucleic acids.

As used herein, “nitrification” refers to the aerobic oxidation of ammonium to nitrate. Nitrification can occur through two subsequent reactions—ammonium oxidation to nitrite (equation 1); and nitrite oxidation to nitrate (equation 2).

NH₄⁺+1.5O₂→NO₂⁻+H₂O+2H⁺ equation 1

NO₂⁻+0.5O₂→NO₃⁻ equation 2

“Comammox” (COMplete AMMonia OXidiser) refers to the complete oxidation of ammonia to nitrate in one organism. Complete oxidation of ammonia to nitrate is represented in equation 3.

NH₄⁺+2O₂→NO₃^{-+H}₂O+2H⁺ equation 3

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “microorganism,” “microorganism host,” “microorganism host cell,” “recombinant host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into a host. It will be appreciated that typically the genome of a recombinant host described herein is augmented through stable introduction of one or more recombinant genes. Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. In some aspects, said recombinant genes are encoded by cDNA. In other embodiments, recombinant genes are synthetic and/or codon-optimized for expression in Nitrospira spp., Nitrosomonas spp., and Nitrosococcus spp., Nitrosospira spp., Nitrobacter spp., Nitrospina spp., and Nitrococcus spp.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast gene. In some embodiments, the gene is endogenous to Nitrospira spp., Nitrosomonas spp., and Nitrosococcus spp., Nitrosospira spp., Nitrobacter spp., Nitrospina spp., and Nitrococcus spp. In some embodiments, an endogenous bacterial gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous gene is deleted. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, Nitrospira spp., Nitrosomonas spp., and Nitrosococcus spp., Nitrosospira spp., Nitrobacter spp., Nitrospina spp., and Nitrococcus spp.

As used herein, the terms “heterologous sequence” and “heterologous coding sequence” are used to describe a sequence derived from a species other than the recombinant host. In some embodiments, the recombinant host is a Nitrospira cell, and a heterologous sequence is derived from an organism other than Nitrospira. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Selection markers are also used for selecting clones that have been transformed with an expression plasmid. Integration of the linear fragments into the genome and the disruption of the gene can be determined based on the selection marker and can be verified by, for example, PCR or Southern blot analysis. Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild-type sequence of a particular protein.

As used herein, the terms “or” and “and/or” is utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” In some embodiments, “and/or” is used to refer to the exogenous nucleic acids that a recombinant cell comprises, wherein a recombinant cell comprises one or more exogenous nucleic acids selected from a group. In some embodiments, “and/or” is used to refer to ammonia oxidation to nitrite and/or nitrite oxidation to nitrate.

### Functional Homologs

Functional homologs of the polypeptides described above are also suitable for use in producing a recombinant host capable of comammox. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of comammox polypeptides. Sequence analysis can involve BLAST, Reciprocal BLAST, or PSI-BLAST analysis of non-redundant databases using an ammonia monooxygenase amino acid sequence, a hydroxylamine dehydrogenase amino acid sequence, and/or a nitrite oxidoreductase amino acid sequence as the reference sequences. Amino acid sequence is, in some instances, deduced from the nucleotide sequence. Those polypeptides in the database that have greater than 40% sequence identity are candidates for further evaluation for suitability as a comammox biosynthetic polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in non-recombinant microorganism capable of comammox, e.g., conserved functional domains. In some embodiments, nucleic acids and polypeptides are identified from transcriptome data based on expression levels rather than by using BLAST analysis.

Conserved regions can be identified by locating a region within the primary amino acid sequence of comammox capable microorganism that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. The information included at the Pfam database is described in Sonnhammer et al., 1998, Nucl. Acids Res., 26:320-322; Sonnhammer et al., 1997, Proteins, 28:405-420; and Bateman et al., 1999, Nucl. Acids Res., 27:260-262. Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 90%, 92%, 94%, 96%, 98%, or 99% amino acid sequence identity.

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between. A percent (%) identity for any candidate nucleic acid or polypeptide relative to a reference nucleic acid or polypeptide can be determined as follows. A reference sequence (e.g., a nucleic acid sequence or an amino acid sequence described herein) is aligned to one or more candidate sequences using generally available computer programs (e.g., Clustal, et al.).

It will be appreciated that functional ammonia monooxygenase (AMO), hydroxylamine dehydrogenase (HAO), and nitrite oxidoreductase (NXR) proteins can include additional amino acids that are not involved in the enzymatic activities carried out by the enzymes. In some embodiments, AMO, HAO, and/or NXR are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding an AMO polypeptide, an HAO polypeptide, and/or an NXR polypeptide can include a tag sequence that encodes a “tag” designed to facilitate subsequent manipulation (e.g., to facilitate purification or detection), secretion, or localization of the encoded polypeptide. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, Conn.). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, AMO, HAO, and/or NXR polypeptides may be altered by domain swapping.

### Recombinant Host

Recombinant hosts can be used to express polypeptides for the oxidation of ammonia and nitrite. A number of bacteria are suitable for use in constructing the recombinant hosts described herein; however, it is also appreciated by one of skill in the art that additional species can be used to express such polypeptides, i.e. yeast, fungi, and archaea. A species and strain selected for use as a comammox capable strain is first analyzed to determine which production genes are endogenous to the strain and which genes are not present. Genes for which an endogenous counterpart is not present in the strain are advantageously assembled in one or more recombinant constructs, which are then transformed into the strain in order to supply the missing function(s).

Typically, the recombinant microorganism is grown in a flask, deep-well plate, or fermentor at a temperature(s) for a period of time, wherein the temperature and period of time facilitate the production of a comammox capable strain. The constructed and genetically engineered microorganisms provided by the invention can be cultivated using conventional fermentation processes, including, inter alia, chemostat, batch, fed-batch cultivations, semi-continuous fermentations such as draw and fill, solid state fermentation, continuous perfusion fermentation, and continuous perfusion cell culture. Levels of substrates and intermediates can be determined by extracting samples from culture media for analysis according to published methods.

After a recombinant microorganism has been grown in culture for the period of time, wherein the temperature, percent oxygen, and period of time facilitate the growth of the recombinant microorganism, the microorganism can be harvested. In some embodiments, the harvested microorganisms may be further processed for optimization of desired traits, for example, but not limited to, shelf-life stabilization, ease of transport, convenience of packaging, and optimization of comammox activity.

Exemplary prokaryotic and eukaryotic species are described in more detail below. However, it will be appreciated that other species can be suitable. For example, suitable species can be in a genus such as Agaricus, Aspergillus, Bacillus, Brocadia, Candida, Crenothrix, Corynebacterium, Eremothecium, Escherichia, Fusarium/Gibberella, Kluyveromyces, Laetiporus, Lentinus, Nitrospira, Nitrosomonas, Nitrosococcus, Nitrosospira, Nitrobacter, Nitrospina, Nitrococcus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Nitrospira moscoviensis, Nitrosomonas europea, Nitrosococcus oceani, Nitrosospira briensis, Nitrobacter vulgaris, Nitrospina gracilis, Nitrococcus mobilis, Phanerochaete chrysosporium, Pichia pastoris, Crenothrix polyspora, Cyberlindnera jadinii, Physcomitrella patens, Rhodoturula glutinis, Rhodoturula mucilaginosa, Phaffia rhodozyma, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia bacteria cells, for example, Escherichia coli cells; Nitrospira bacteria cells; Nitrosomonas bacteria cells; Nitrosococcus bacteria cells; Nitrosospira bacteria cells; Nitrobacter bacteria cells; Nitrospina bacteria cells; Nitrococcus bacteria cells; Lactobacillus bacteria cells; Lactococcus bacteria cells; Cornebacterium bacteria cells; Acetobacter bacteria cells; Acinetobacter bacteria cells; or Pseudomonas bacterial cells.

As indicated, nucleic acid molecules of the present invention may be in the form of RNA, such as 16S rRNA and mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

### Nitrospira spp.

Nitrospira is a genus of bacteria in the phylum Nitrospirae. Nitrospira members are chemolithoautotrophic nitrite-oxidizing bacteria. Nitrospira-like bacteria take up inorganic carbon (like HCO3- and CO2) as well as pyruvate under aerobic conditions. Recently, members of Nitrospira have been discovered to perform complete nitrification (see Daims et al., Nature, 2015 (528) 504-509; van Kessel et al., Nature, 2015 (528) 555-559). Nitrospira is found throughout the world and is a diverse group of nitrite-oxidizing bacteria. Members have been found in terrestrial and limnic habitats, marine waters, deep sea sediments, sponge tissue, geothermal springs, drinking water distribution systems, corroded iron pipes, and wastewater treatment plants.

### Nitrosomonas spp.

Nitrosomonas is a genus of ammonia-oxidizing proteobacteria. Nitrosomonas are rod-shaped chemolithoautothrophs with an aerobic metabolism. Nitrosomonas are among the ammonia-oxidizing bacteria reported to have health benefits for humans as the oxidation of ammonia produces nitric oxide. Nitric oxide is known to be a part of physiological functions such as vasodilation, skin inflammation and wound healing.

### Nitrosococcus spp.

Nitrosococcus is a genus of ammonia-oxidizing proteobacteria. Nitrosococcus oceani was the first reported member and was discovered by isolation from open ocean water in 1962 by Stanley Watson.

### Nitrosospira spp.

Nitrosospira is a genus of ammonia-oxidizing proteobacteria.

### Nitrobacter spp.

Nitrobacter is a genus of nitrite-oxidizing chemoautotrophic proteobacteria.

### Nitrospina spp.

Nitrospina is a genus of nitrite-oxidizing chemolithoautotrophic proteobacteria that have been exclusively found in marine environments.

### Nitrococcus spp.

Nitrococcus is a genus of nitrite-oxidizing chemolithoautotrophic proteobacteria.

### Chrenothrix spp.

Crenothrix is a genus of methane-oxidizing proteobacteria which encodes a phylogenetically unusual articulate methane monooxygenase (PMO) that is more closely related to the amoA of betaproteobacterial ammonia oxidizers than to the pmoA of other methanotrophs (see Stoecker et al., PNAS, 2006 (103)(7) 2363-2367).

### Brocadia spp.

Brocadia is a genus of anaerobic chemolithoautotrophic bacteria that belong to the order of Planctomycetes.

Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

### Comammox Biosynthetic Nucleic Acids

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In many cases, the coding sequence for a polypeptide described herein is identified in a species other than the recombinant host, i.e., is a heterologous nucleic acid. Thus, if the recombinant host is a microorganism, the coding sequence can be from other prokaryotic or eukaryotic microorganisms, from plants or from animals. In some case, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. However, it will be understood by one having skill in the art that nucleotide sequences of accessory genes, non-comammox nucleic acids, either exogenous or endogenous, are linked to the desired comammox nucleic acids, at positions where the native sequence would be found, e.g., cytochrome c sequences flanking AMO, HAO, and/or NXR. In certain embodiments, the genes allowing for comammox are localized on a single contiguous genomic fragment, which can also contain general housekeeping genes.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

Ammonia monooxygenase refers to a nucleotide sequence, a gene, a nucleic acid sequence, a protein, and/or an enzyme that performs ammonia oxidation. In some embodiments, the nucleotide sequence of a nucleic acid encoding an ammonia monooxygenase (AMO) polypeptide is set forth in SEQ ID NOs: 2, 4, and/or 6. In some aspects, the nucleic acid encoding the AMO polypeptide has at least 70% identity to the nucleotide sequence set forth in SEQ ID NOs: 2, 4, and/or 6, at least 80% identity to the nucleotide sequence set forth in SEQ ID NOs: 2, 4, and/or 6, at least 95% identity to the nucleotide sequence set forth in SEQ ID NOs: 2, 4, and/or 6. In some embodiments, the amino acid sequence of a AMO enzyme is set forth in SEQ ID NOs: 2, 4, and/or 6. In some embodiments, a host cell comprises one or more copies of one or more nucleic acids encoding an AMO polypeptide. In some embodiments, there are multiple AMOs in one cell. It will be understood by one in the art that while the multiple AMOs can be repeats of the same AMO, the multiple AMOs can be discrete sequences. AMO contains three different subunits, alpha (AmoA), beta (AmoB), and gamma (AmoC). In some embodiments, the three different subunits are encoded by a single amoCAB gene cluster and comprise additional amo genes at other genomic loci.

Hydroxylamine oxidoreductase (HAO) refers to a nucleotide sequence, a gene, a nucleic acid sequence, a protein, and/or an enzyme which performs ammonia oxidation. HAO is also called and can be used interchangeably with hydroxylamine dehydrogenase. In some aspects, the nucleic acid encoding the HAO polypeptide has at least 70% identity to the nucleotide sequence set forth in SEQ ID NOs: 8 and/or 10, at least 80% identity to the nucleotide sequence set forth in SEQ ID NOs: 8 and/or 10, at least 95% identity to the nucleotide sequence set forth in SEQ ID NOs: 8 and/or 10. In some embodiments, the amino acid sequence of a HAO enzyme is set forth in SEQ ID NOs: 8 and/or 10. In some embodiments, a host cell comprises one or more copies of one or more nucleic acids encoding an HAO polypeptide. In some embodiments, there are multiple HAOs in one cell. It will be understood by one in the art that while the multiple HAOs can be repeats of the same HAO, the multiple HAOs can also be discrete sequences.

Nitrite oxidoreductase (NXR) refers to a nucleotide sequence, a gene, a nucleic acid sequence, a protein, and/or an enzyme that performs nitrite oxidation. NXR is the key enzyme for nitrite oxidation, the last reaction in the nitrification process. NXR is bound to the inner cytoplasmic surface of the bacterial membrane and contains multiple subunits, iron-sulfur centers and a molybdenum cofactor. The NXR subunits include alpha, beta, and gamma. In some aspects, the nucleic acid encoding the NXR polypeptide has at least 70% identity to the nucleotide sequence set forth in SEQ ID NOs: 12 and/or 14, at least 80% identity to the nucleotide sequence set forth in SEQ ID NOs: 12 and/or 14, at least 95% identity to the nucleotide sequence set forth in SEQ ID NOs: 12 and/or 14. In some embodiments, the amino acid sequence of a NXR enzyme is set forth in SEQ ID NOs: 12 and/or 14. In some embodiments, a host cell comprises one or more copies of one or more nucleic acids encoding an NXR polypeptide. In some embodiments, there are multiple NXRs in one cell. It will be understood by one in the art that while the multiple NXRs can be repeats of the same NXR, the multiple NXRs can also be discrete sequences.

In some embodiments, the proteins allowing the microorganism the ability to perform comammox are phylogenetically affiliated with proteins having differing functions in other microorganisms. In example, an AMO protein may have over 95% homology to methane monooxygenase (PMO) of Crenothrix polyspora. Methane monooxygenase is in the family of oxidoreductases and has the ability to oxidize alkanes into primary alcohols, for example PMO catalyzes the conversion of methane to methanol. A representative sequence of PMO is shown in SEQ ID NOs: 16, 18, and 20.

In some embodiments, urease inhibitors are added to the composition of bacteria as disclosed. Urease inhibitors prevent the production of ammonia from nitrogen by urease enzymes. Mechanically speaking, urease inhibitors can be classified into two broad categories, substrate structural analogs and phosphodiamidates—inhibitors which affect the mechanism of reaction. Structurally speaking, there are four families of urease inhibitors: 1) thiolic compounds; 2) hydroxamic acid and derivatives; 3) phosphorodiamidates; and 4) ligands and chelators of nickel. Of these, phosphorodiamidates are the most effective of the groups.

### Fabric

Fabric, as used herein, represents a cloth-like object which is typically produced by weaving or knitting textile fibers. While fabric includes articles of clothing, fabric is not limited to articles of clothing. Fabric can include both natural, synthetic, and a combination of natural and synthetic fibers. Examples of fabric that may be treated by the composition of this disclosure include, but are not limited to, carpets, rugs, drapes/curtains, automobile upholsteries, furniture upholsteries, home décor fabrics, denims, corduroys, polyesters, flannels, fleeces, cottons, silks, elastane, linens, lyocells, rayons, nylons, polyurethanes, viscoses, polyamides, acetates, tweeds, velour, wool, and others. A person having skill in the art will recognize what is considered a fabric.

Denim blue jeans are known for their broad inclusion in wardrobes worldwide. Once the daily standard of workers, blue jeans have become a fashion mainstay. Certain fashion-conscious consumers prefer their denim to possess distressed or “vintage” aesthetics, including “fades” and “creases.” Other consumers merely prefer the feel of a broken-in pair of jeans, a feel that disappears when the denim is washed.

Some consumers, especially those with a preference for “raw” or “unwashed” denim, prefer to obtain distressed and vintage looks through personal use and corollary wear. Others prefer their denim possess these characteristics from the outset, and manufacturers have devised processing techniques to “finish” denim so that it appears distressed at the point of purchase.

Additional embodiments are directed toward the use of the disclosed composition on athletic apparel. Athletic apparel has become more specialized over the past decade with the introduction to “technology” built in to the fabric. Some such technological fabrics comprise properties like moisture-wicking, specialty base layers, breathability, vented fabrics, lightweight fabrics, compression, and others. Many of these fabrics are synthetic, while others are natural, and some are hybrid of natural and synthetic fibers. Such specialty items can be waterproof and windproof outerwear which is also breathable.

Additional embodiments are directed toward the use of the disclosed composition and methods of use on athletic/sporting equipment. In this context, athletic or sporting equipment can be items that are worn by an athlete and serve a benefit to the athlete. Examples of sporting equipment are, but not limited to, helmets, protective padding, gloves, masks, and hats. As will be recognized by one of skill in the art, these “categories” of athletic apparel, apparel, athletic equipment, and fabrics are not always defined, and cross-over from one category to another is not only common, but often expected.

### EXAMPLES

The Examples which follow are illustrative of specific embodiments of the invention, and various uses thereof. They set forth for explanatory purposes only, and are not to be taken as limiting the invention.

### Example 1

### Treatment of Denim Fabric with Composition of Bacteria

A composition comprising Nitrospira bacteria was mixed with water in about a one to twenty-five (1:25) ratio. The mixture was placed into a commercially available household spray bottle and sprayed onto the surface of a denim swatch containing malodor from use. The diluted composition was applied to the denim swatch five times, by way of five sprays from the household spray bottle. A second denim swatch from the same denim containing malodor from the same use was left untreated as a control. The denim swatches were each placed in separate bags and remained untouched for about 48 hours. After about 48 hours, the swatches were removed from their respective bags and tested through a blinded panel for malodor. The malodor significantly diminished in the treated denim swatch but was still evident in the untreated swatch.

The same experiment will be run for measurements of quantifiable ammonia levels. In quantifying ammonia, methods of quantifying ammonia known in the art will be utilized. For example, the sampling and analytical method (OSHA Method ID-188, January 2002) developed by the United States Department of Labor, Occupational Safety and Health Administration.

### Example 2

### Treatment of Denim Fabric with Composition of Bacteria

A composition comprising Nitrospira bacteria and a urease inhibitor was mixed with water in about a one to twenty-five (1:25) ratio. The mixture was placed into a commercially available household spray bottle and sprayed onto the surface of a denim swatch containing malodor from use. The diluted composition was applied to the denim swatch five times, by way of five sprays from the household spray bottle. A second denim swatch from the same denim containing malodor from the same use was left untreated as a control. The denim swatches were each placed in separate bags and remained untouched for about 48 hours. After the about 48 hour period, the swatches were removed from their respective bags and tested through a blinded panel for malodor. The malodor had disappeared in the treated denim swatch but was still evident in the untreated swatch.

The same experiment will be run for measurements of quantifiable ammonia levels. In quantifying ammonia, methods of quantifying ammonia known in the art will be utilized. For example, the sampling and analytical method (OSHA Method ID-188, January 2002) developed by the United States Department of Labor, Occupational Safety and Health Administration.

### Example 3

### Concentration Gradient Testing for the Treatment of Denim Fabric with Composition of Bacteria

A composition comprising an mixture of bacteria capable of complete nitrification are mixed with water in concentration gradient ratios ranging from about 1:200 to 1:1. The diluted compositions will be placed into a commercially available household spray bottle and sprayed onto the surface of a denim swatch containing malodor from use. Each concentration of the composition admixture will be applied to a denim swatch five times, by way of five sprays from the household spray bottle. A denim swatch from the same denim containing malodor from the same use is left untreated as a control. The denim swatches are each placed in separate bags and remain untouched for 48 hours. After the 48 hour period, the swatches are removed from their respective bags and tested for malodor. The malodor is tested in a way known in the art that produces quantifiable levels of ammonia. In some cases, the control swatch ammonia quantification will be normalized to the quantification at time-point zero and the quantification at time-point 48 hours. The experimental swatch ammonia quantifications can be normalized to the normalized control quantification.

### Example 4

### Time-Series Testing for the Treatment of Denim Fabric with Composition of Bacteria

A composition comprising an mixture of bacteria capable of complete nitrification are mixed with water in a ratio of about 1:25. The diluted composition will be placed into a commercially available household spray bottle and sprayed onto the surface of a denim swatch containing malodor from use. The composition admixture will be applied to a series of denim swatches five times, by way of five sprays from the household spray bottle to each swatch. A series of denim swatches from the same denim containing malodor from the same use are left untreated as controls. The denim swatches, both treated and untreated, are each placed in separate bags and remain untouched for a gradient of time points. The time points ranging from about 6 hours to about 96 hours. After each time point, a control swatch and a treated swatch are removed from their respective bags and tested for malodor. The malodor is tested in a way known in the art that produces quantifiable levels of ammonia.

### Example 5

### Treatment of Sports Equipment with Composition of Bacteria

A composition comprising a mixture of bacteria capable of complete nitrification is mixed with water in a ratio of about 1:25. The mixture is then placed into a commercially available household spray bottle. The mixture is then sprayed onto the surface and into the insides of one goalie glove from a pair of gloves worn by a soccer player. The other goalie glove is left untreated. The gloves are each placed into bags and allowed to sit at ambient temperature for about 48 hours. After about 48 hours, the gloves are removed from their respective bags and tested for measurements of quantifiable ammonia levels.

One of skill in the art will readily appreciate that the present invention is well adapted to carry out the objects and obtain the ends and advantages mentioned, as well as those inherent therein. The present examples along with the methods, procedures, and specific compositions described herein are presently representative of preferred embodiments, are exemplary, and are not intended as limitations on the scope of the invention. Changes therein and other uses will occur to those skilled in the art which are encompassed within the spirit of the invention as defined by the scope of the claims.

TABLE 1
Sequences disclosed herein.

| SEQ | |
|---|---|
| ID | |
| NO: | Description |
| 1 | DNA sequence of ammonia monooxygenase subunit A, |
| | Nitrospira inopinata* |
| 2 | Protein sequence of ammonia monooxygenase subunit A, |
| | Nitrospira inopinata |
| 3 | DNA sequence of ammonia monooxygenase subunit B, |
| | Nitrospira inopinata* |
| 4 | Protein sequence of ammonia monooxygenase subunit B, |
| | Nitrospira inopinata |
| 5 | DNA sequence of ammonia monooxygenase subunit C, |
| | Nitrospira inopinata* |
| 6 | Protein sequence of ammonia monooxygenase subunit C, |
| | Nitrospira inopinata |
| 7 | DNA sequence of hydroxylamine reductase subunit A, |
| | Nitrospira inopinata* |
| 8 | Protein sequence of hydroxylamine reductase subunit A, |
| | Nitrospira inopinata |
| 9 | DNA sequence of hydroxylamine reductase subunit B, |
| | Nitrospira inopinata* |
| 10 | Protein sequence of hydroxylamine reductase subunit B, |
| | Nitrospira inopinata |
| 11 | DNA sequence of nitrite oxidoreductase subunit A, |
| | Nitrospira inopinata* |
| 12 | Protein sequence of nitrite oxidoreductase subunit A, |
| | Nitrospira inopinata |
| 13 | DNA sequence of nitrite oxidoreductase subunit B, |
| | Nitrospira inopinata* |
| 14 | Protein sequence of nitrite oxidoreductase subunit B, |
| | Nitrospira inopinata |
| 15 | DNA sequence of methane monooxygenase/ammonia |
| | monooxygenase subunit A, Crenothrix polyspora |
| 16 | Protein sequence of methane monooxygenase/ammonia |
| | monooxygenase subunit A, Crenothrix polyspora |
| 17 | DNA sequence of methane monooxygenase/ammonia |
| | monooxygenase subunit B, Crenothrix polyspora |
| 18 | Protein sequence of methane monooxygenase/ammonia |
| | monooxygenase subunit B, Crenothrix polyspora |
| 19 | DNA sequence of methane monooxygenase/ammonia |
| | monooxygenase subunit C, Crenothrix polyspora |
| 20 | Protein sequence of methane monooxygenase/ammonia |
| | monooxygenase subunit C, Crenothrix polyspora |

*Denotes coding sequence on ENR4 genome assembly NiCH1, chromosome: 1.

| SEQ ID NO: 1 | |
|---|---|
| | atg tttagaacgg atgaaataat caaagccgcc aagttgcctc cagagggagt |
| 1789081 | ggcgatgtcg cggcacattg attacattta ctttattcct attttgttcg tgaccatcat |
| 1789141 | cggaactttt cacatgcaca cggctttgtt gtgcggtgac tgggatttct ggttggattg |
| 1789201 | gaaggatcgg cagtggtggc cgattgtgac tcccatcaca acaattacct tctgcgcagc |
| 1789261 | ccttcaatac tataactggg tcaattatcg tcagccgttt ggggcaacga taaccatttt |
| 1789321 | agcgttaggt gccggaaaat gggttgcggt ttacacctct tggtggtggt ggtccaacta |
| 1789381 | tccgccaaat ttcgtcatgc cggccacgtt gcttcctagc gccttggttc ttgatttcac |
| 1789441 | cttgttgcta actagaaact ggactttgac cgcagtgatc ggggcctgga tgtacgcgat |
| 1789501 | tttgttctat ccgagcaatt ggcctatctt tgcttacagc catactccgc ttgtggtgga |
| 1789561 | tgggaccttg ctttcatggg ccgactatat gggctttatg tatgtgcgga ccggaactcc |
| 1789621 | tgaatatatc cgtatgattg aagttgggtc gctgcggacg ttcggtgggc acagcacgat |
| 1789681 | gatttcctcg ttctttgctg cattcgcctc ttcattgatg tacatcctgt ggtggcagtt |
| 1789741 | tggaaagttt ttctgcacgt cctatttcta cttcacggat gacaagaagc gaacgaccaa |
| 1789801 | agtttacgat gtctttgcct atgcaacatt ggctcacgcg gataaggcca aactctctgg |
| 1789861 | ggggaaagca tga |
| SEQ ID NO: 2 | |
| 1 | mfrtdeiika aklppegvam srhidyiyfi pilfvtiigt fhmhtallcg dwdfwldwkd |
| 61 | rqwwpivtpi ttitfcaalq yynwvnyrqp fgatitilal gagkwvavyt swwwwsnypp |
| 121 | nfvmpatllp salvldftll ltrnwtltav igawmyailf ypsnwpifay shtplvvdgt |
| 181 | llswadymgf myvrtgtpey irmievgslr tfgghstmis sffaafassl myilwwqfgk |
| 241 | ffctsyfyft ddkkrttkvy dvfayatlah adkaklsggk a |
| SEQ ID NO: 3 | |
| | a tgaacgtcaa acacgtcttc aagctgtgga tgctgggatt ctgcggagtg |
| 1789921 | gcgacgttgg cgttcacgcc ggtgtttgat gctgctccag ttcttgctca cggggagcgt |
| 1789981 | tcgcaagaac cgtttctgcg gatgcgcacc gtgaattggt atgacactga atgggtgggg |
| 1790041 | aaaagcactg cggtaaatga tgttacatac atgaggggca agtttcatct gtctgaagac |
| 1790101 | tggcctcgtg cggtagtgaa accccatcga acgttcgtca atgtcggctc tcctagctcc |
| 1790161 | gtctttgtgc ggttaagcac gaaggttggt ggggtgccga tgtttgtgtc tggtcctatg |
| 1790221 | gaaatcgggc gtgattatga atatgagatc acgttgaagg cgagacttcc tggacatcat |
| 1790281 | cacattcacc ctatgttttc tgttaaagag gctggtccca ttgccggacc gggtgggtgg |
| 1790341 | atggatatca cgggccgata cgctgatttt acaaacccga tcaagactct gacgggggaa |
| 1790401 | acatttgact cggaaacaga gggtgggatg accggaatta tgtggcatat attctgggca |
| 1790461 | tctgttgcct tgttctgggt gggttggttc atggttcgcc cgatgtactt gattcgggct |
| 1790521 | cgtgtgcttg cggcttatgg tgatgaactt ctgttggatc cggttgatcg caagctcgca |
| 1790581 | ataggtcttc tcgtatttac ggtggcggtt gtcactatcg gttatctcgc tgcggaggcg |
| 1790641 | aagcatccta ttaccgtgcc cctgcaggct ggtgaagcaa aggttaaacc gcttcctata |
| 1790701 | aaaccgaatc cattggtggt tgaagtcacc cacgccgaat atgacgtgcc gggtcgtgct |
| 1790761 | cttcgtatga cggttcacgc cactaacaat gggactgagc ctgtcagtat cggtgaattc |
| 1790821 | acaacggctg gtattcgatt tacaaataaa gtaggagcag cgaagctcga tccgaactat |
| 1790881 | ccacaggagc ttattgctac agccggactg accatggata atgaggctcc gatacagccg |
| 1790941 | ggtcagactg ttgacattca catagaatca aaggatgttc tatgggaggt tcagcggctg |
| 1791001 | gttgacattc ttcacgatcc ggatcagcgg tttgctgggt tgttgatgtc atggactgaa |
| 1791061 | tcgggagaac gtcttattaa ccccgtgtgg gctcctgtgc ttcctgtctt tacacgattg |
| 1791121 | ggagcataa |
| SEQ ID NO: 4 | |
| 1 | mnvkhvfklw mlgfcgvatl aftpvfdaap vlahgersqe pflrmrtvnw ydtewvgkst |
| 61 | avndvtymrg kfhlsedwpr avvkphrtfv nvgspssvfv rlstkvggvp mfvsgpmeig |
| 121 | rdyeyeitlk arlpghhhih pmfsvkeagp iagpggwmdi tgryadftnp iktltgetfd |
| 181 | seteggmtgi mwhifwasva lfwvgwfmvr pmylirarvl aaygdellld pvdrklaigl |
| 241 | lvftvavvti gylaaeakhp itvplqagea kvkplpikpn plvvevthae ydvpgralrm |
| 301 | tvhatnngte pvsigeftta girftnkvga akldpnypqe liatagltmd neapiqpgqt |
| 361 | vdihieskdv lwevqrlvdi lhdpdqrfag llmswtesge rlinpvwapv lpvftrlga |
| SEQ ID NO: 5 | |
| | ctagta gcctgctttg |
| 734761 | gcgttggggt tgacctggct ggggaacgga tccaggatgc tcttgggcgc cccgttccaa |
| 734821 | atcacatccg ccaagttcga catgcggctc acaatctgcg ccgccacgcc gccggccgcc |
| 734881 | ccgaacaacc cgcaccagcc caacgtcaca aagccccaat gcaacggcgc cgcaaacaac |
| 734941 | tcgtccacaa accaaaacgc atggccccac tcattgagcc ccacgttcgg caaaatgaac |
| 735001 | atcggcccca ccaccgccgc caccaacgga aacgacgtcg cctggctata caacggcaac |
| 735061 | cgcgtctgcg catacagata actcgagacc ccgcacgtaa tgtacaacgg gaacgtcccg |
| 735121 | taaaacgcca caatgtgact ggccgtaaaa ctcgtgtccc gaatgatcac ctgatgccac |
| 735181 | gccgcatcct gctccaacgt gtagctgccc gcataataga ccccccacac gtagcaggcc |
| 735241 | aaccacccca tccagtaaaa ataccgcttc aactccgtct tcggatccaa attcgccaaa |
| 735301 | ttccgatccc gcgtcaccca aatccacccc accgaaatcg caaaaaataa cgcattggcc |
| 735361 | acaatgttaa accgccataa ccccatccac accgcgtcaa actccggcgt catcgagtcc |
| 735421 | aacccgtgcg aatacccaaa cgtccgctga tacaacaccc aaaaaatccc aatcgccaac |
| 735481 | atcgcaaacc acccaatctt ccacggccgc gaatcatacc actgcgaaat gtcatacccc |
| 735541 | cgctctgccg ccat |
| SEQ ID NO: 6 | |
| 1 | maaergydis qwydsrpwki gwfamlaigi fwvlyqrtfg yshgldsmtp efdavwmglw |
| 61 | rfnivanalf faisvgwiwv trdrnlanld pktelkryfy wmgwlacyvw gvyyagsytl |
| 121 | eqdaawhqvi irdtsftash ivafygtfpl yitcgvssyl yaqtrlplys qatsfplvaa |
| 181 | vvgpmfilpn vglnewghaf wfvdelfaap lhwgfvtlgw cglfgaaggv aaqivsrmsn |
| 241 | ladviwngap ksildpfpsq vnpnakagy |
| SEQ ID NO: 7 | |
| | tcaacgatc tttcctctct cgtcgacgcc agccagcaat tgccaacgta ccggccagca |
| 58621 | tcatacctcc gcccaacccc ccgatcgaca gcttcccgcc cgggccatcg agatcaagca |
| 58681 | gagagtgctt gcgctcgctc tcaagcttat cgacacgggc caacaacttt tgtgtttcct |
| 58741 | taagccgagt gtcatcgtcc atgatttcca cataggcccg attcatcgca gcccaaccaa |
| 58801 | ccgtataggt atatccccaa tactggtgag ccaagctgac gtgcaattga accaggtgat |
| 58861 | cctccgccat ttcgaacagc ttgagctcgt tggccgccgg gttatttccc tttgaccagt |
| 58921 | aaatctggaa gaactgctca aagccgtcct tcaccggagg aggcggtgcg ggccggttgg |
| 58981 | ttttttgccc cgtcaacaac ccggccttgt actgctcttc gactacgtga tgagcctcgt |
| 59041 | cgtacttatc aagaccggag tacgtacctt tatccatgaa ctccatccag gcgcgggcgt |
| 59101 | aactttccga gtgacagttc gtacaggttt taacccacgc atcgagccgc ttttccgacc |
| 59161 | aatcggtcga gatattctct cggatgccag gaacgaacgg atagttggcc catcgaacct |
| 59221 | tgcgcaccac gttgtgggtt atcttacctt gatattccat gtggcagaat tggcatgtcg |
| 59281 | gggcactcaa tccacccttg gcaatggctt ctttaatggg aatattgaaa ttccacttat |
| 59341 | ctttgtctct ctgatacttc aatccatgct tggagaggga gtaggcctcc cagttgttgt |
| 59401 | gatcggcccc actgtgacac tgagcgcaat tttccggctt gcgcgattcc gcgactgaga |
| 59461 | attcatgacg cgagtgacaa gtatcgcact tgttctgatt gacgtgacaa ccggtacagc |
| 59521 | catcggcgat ttctctttga ggcatcccgg catagacgtc cacttccacg ttagccttat |
| 59581 | aatccaaggc atgcgaagga cgccccttgg gccactgatc ttttggccat atgatggtgt |
| 59641 | cacgctccga ttctcgttca gcgaattcct gaagatggca cgtaccgcag gtgttcgccg |
| 59701 | tcgccagctt aatgtccttg cgatgatcgg ccttcccttt ggcattgatt tcaaagtgac |
| 59761 | aatcaatgca cccaacttct tttagctgct cccccttgcc caacttgccc atcgaccgaa |
| 59821 | ggttttcctc gatttgctca agcttcgcct tcttgtaata ggtctcatcc ttgggcgtca |
| 59881 | gtttacggat cttatccaga ttggcatgcg tgctgcgctt ccacgcagcc acccaccccg |
| 59941 | gcgattcatc cgtatgacat ttgacgcact gctcacggct tgcgacttcc ttgactgcct |
| 60001 | gcgggggctt atagaacgtc gtgggatcga aatacttact gaaagtgacg ggctcccagt |
| 60061 | actgaccgta aattcccttt ccggcccctt gctcaggatc gaggtacctt ttcaccagag |
| 60121 | cctcgtacag ctcctttgga gaagccgaac ggtcgatctt cagtgcctca taagtttcct |
| 60181 | tcggcaccgt cgggaagtcc gcttgcgccg gtgcggccag taaaacgccg cagacgagca |
| 60241 | tcacaaactt ctgcgcaaac ttgctgctca t |
| SEQ ID NO: 8 | |
| 1 | msskfaqkfv mlvcgvllaa paqadfptvp ketyealkid rsaspkelye alvkryldpe |
| 61 | qgagkgiygq ywepvtfsky fdpttfykpp qavkevasre qcvkchtdes pgwvaawkrs |
| 121 | thanldkirk ltpkdetyyk kakleqieen lrsmgklgkg eqlkevgcid chfeinakgk |
| 181 | adhrkdikla tantcgtchl qefaereser dtiiwpkdqw pkgrpshald ykanvevdvy |
| 241 | agmpqreiad gctgchvnqn kcdtchsrhe fsvaesrkpe ncaqchsgad hnnweaysls |
| 301 | khglkyqrdk dkwnfnipik eaiakgglsa ptcqfchmey qgkithnvvr kvrwanypfv |
| 361 | pgirenistd wsekrldawv ktctnchses yarawmefmd kgtysgldky deahhvveeq |
| 421 | ykaglltgqk tnrpappppv kdgfeqffqi ywskgnnpaa nelklfemae dhlvqlhvsl |
| 481 | ahqywgytyt vgwaamnray veimdddtrl ketqkllarv dkleserkhs lldldgpggk |
| 541 | lsigglgggm mlagtlaiag wrrrerkdr |
| SEQ ID NO: 9 | |
| | tcagt tttgccgttg aatctgctca |
| 57541 | gcagaaggga ttttgtatat ccaatatccc ccttctttat gtattaattg caacgcatca |
| 57601 | agatccatcg gtctaaagtt ggaaaacggc aacattttcg ccaacagtat gttgctggct |
| 57661 | ccgctttctc ggaggaaata agcacgcact tgcttctcgg agagagactg aagggtatag |
| 57721 | cttgtgtagt cgttgtttgt cagccaggct ttcacctgcc cgctgagtcc atggacgttt |
| 57781 | cctgtcaagg gaaaatcctt aaatgcaaca tctatgcgat caggtcgcat cagtcccaac |
| 57841 | ttataaatgt cgctcacatg cacgaccacg tatgcttctc gcgaaccaac cagctcgcgc |
| 57901 | aacattgcgg caccttgatg agggtacgcc atgagagctt caataaatcg gttaaactgc |
| 57961 | gcgcgttctt ccggagaagc ctgcttcccc caaaacttct gctcgtactg ttcaatcgct |
| 58021 | tcaaatcggt ctctccagta cgacggggcg atgaccggca agccaagata agcggtgaag |
| 58081 | agcgtcggac gatccgacaa aagggccagt tttctcgaag tatcccacca tgcgacgatg |
| 58141 | acggcatcgt tcggcacatg tttggcgatg gcggacgata cggcgatcgt ttctgaaagc |
| 58201 | ccttcttcca tggataaaat cggttctgga aacttatttt ccacatacag aagaacagga |
| 58261 | tcgaaaccgg agcggtttgc gcgatagaca aaggcgagcg gttgatcgat tgcgtccact |
| 58321 | tgtacttcga gcttaccgat tgtcagatat ggataatttt ccaatcccag gttagggaat |
| 58381 | ttatccgggc cgccctcctc caccacacga tagtgatagg gcggtggttc cggttgaaac |
| 58441 | cacaaataga caaaccatcc tactaaaaaa aggcctcccg ccac |
| SEQ ID NO: 10 | |
| 1 | magglflvgw fvylwfqpep ppyhyrvvee ggpdkfpnlg lenypyltig klevqvdaid |
| 61 | qplafvyran rsgfdpvlly venkfpepil smeeglseti avssaiakhv pndavivaww |
| 121 | dtsrklalls drptlftayl glpviapsyw rdrfeaieqy eqkfwgkqas peeraqfnrf |
| 181 | iealmayphq gaamlrelvg sreayvvvhv sdiyklglmr pdridvafkd fpltgnvhgl |
| 241 | sgqvkawltn ndytsytlqs lsekqvrayf lresgasnil lakmlpfsnf rpmdldalql |
| 301 | ihkeggywiy kipsaeqiqr qn |
| SEQ ID NO: 11 | |
| | ttat actttaatct tgatgtgctc acctttcagc cacttgatca |
| 838381 | taaattcatt ttcctggccc ggcgtgaacc ctgtccgcac cggttcccac gggccacgtg |
| 838441 | ccccaatgcc gccatcctcc gccttcgtga tacggataag gcattctttc gggacggtgt |
| 838501 | tgatggcatg gtgatcgact tgatagcccc acttgaactt ccaggcaatg gcatgtttgc |
| 838561 | ctggcaatga gtcggtttga tgcatcggca tcagccagtt ccgcgtaaac gattgctgac |
| 838621 | acccatatct aaagtttgac tgatagccgg tatcgatggc aatcgcacgt ccatcaggtc |
| 838681 | ttgtttcgtg cccttttacg gactttggcg tcgccacgaa cggagcgtgc ttggccatcg |
| 838741 | tcacgtggta gggataggcg gggttgtact tggcccgaat catcagccga gcgaccttgt |
| 838801 | aataggggtc actgggcttc caaccacggt aaggccgatc caccggattt ccatcgacat |
| 838861 | aaacgtagtc gccatcgttg atcccacgat ccttcgcagc ctggggattg atgtgaagct |
| 838921 | gatgttctcc cacgcccgga gtccgtttat ccatacgata gggatcgccg aagttcgact |
| 838981 | catagatctg cacccagtcg ttcaccgacc actggctgtg aacacggtga cgagtctttg |
| 839041 | gcgtaacaca gtagaactgg taccccttct cccacagcgg attactgtgc cgcttgatct |
| 839101 | catcccacga gagcttaatg ttacggacgg ttttgtcatc atggtgctga gccgtgatgg |
| 839161 | gtatcccgta gtcatcaggc cgcacatagg gattggttgt aaagatggca ttgggcaggt |
| 839221 | acggagttgc ctctggaccc tcccgatgcg aaatgaaatt ttctccatac tcgatggctt |
| 839281 | cggcttccgg tcgatagttt tcatatcttc cgcttcgcgt ccacatgggt ttggactcgt |
| 839341 | tggtctcttc ccagaatggg tggcgcggat aggttctcac catgaccatc cacccctttt |
| 839401 | cagacttgag catgacgtcc gccgagtagc cgtagaacgt gcttgaggcg tcaagcattc |
| 839461 | gctgcgcata gacatcaaca cgattggcat agaccatcgc gaagtaatct ttcatccgtt |
| 839521 | tatcgccggt catgtcggac agtttggctg ccactccggc aaacgtatcg aggtcgttac |
| 839581 | gggtgtcata gaggggcctg attccgccct tccagatctg aacccatggg ttggataccg |
| 839641 | tgatggtcat ttccggatac gtaaattcca tccaagagtt gcaagcaaac gcgatatccg |
| 839701 | catggttgat gtctgatgtc atttcgatgt cttgagtgat cagacattca atgttcggat |
| 839761 | ccacgttttt gaccatgtca tagtggtgct tggcgttatt gaccacgttg acgtttgtca |
| 839821 | cccagcggaa tttactcgga gtcggcatgt gcgtctttcc ggtaaacacc ttgcgtccat |
| 839881 | acttaggtgt attgacgatc aaagccgtgt caccgtgatt ccagtacccg acttcttccc |
| 839941 | cgtaataata ggacctcgta tggatctcct ttccgtgcgc attgggatcc aacgtgatat |
| 840001 | tgaacggatc ttctcccgtg tgaacactga gtccggcccc cgaccatggc gtggcagtcc |
| 840061 | atgcgccggc cttatagttt ccggcccaag tatgctgacc ggtcccgaac ttgccaacgt |
| 840121 | tccccgtgat aatcagcacc atcgcagccc cacgggcgtt gatggtctga tggaagtagt |
| 840181 | ggcacgtgcc ttcgccattg tgaatcgcgg ccggtttaat agtgcccgaa tcacgagccc |
| 840241 | atcgcacaat aaggtctttc ggcgttcgcg tgatctggtg aaccgtatca agatcataat |
| 840301 | cctggaaatg caccatgtac atttgccata taggcatggc atcaatttca cgcccgttca |
| 840361 | gcaatttgac tctgtacgta ccggtcaggg ccgcatcgat accgctgttc acatagtgcc |
| 840421 | atccgacctg ctctcgatgg aggggaacag cctgcttttt attgaggtcc cacaccatca |
| 840481 | tcccgcctaa acgctgaatc tgctcgggct tgagcgactg aattctcccc gaatagcttt |
| 840541 | ttgaaaaatc aggaaatttg taatcaggaa tgacatcgcg cgggtccaaa tattggagcg |
| 840601 | tgtccgttcg cacaagaatt ggggcatcgg taaaggattt cagaaaatca acgtcgtgca |
| 840661 | tgttctcatc gacgataatc ttcatcgccc ccaaaaagag cgcgccatca gactgcggac |
| 840721 | gaagcggcat ccagtaatca gcccgatagg cagtggggtt gtattcggga gtgatcacaa |
| 840781 | caaccctcgc gcctcgctcg atacattcga gcttccaatg ggcctccggc atcttgtttt |
| 840841 | caacaaagtt ctttccccag ctcgtgttca atttagaaaa acgcatgtcg gataggtcaa |
| 840901 | cgtcagaccc ctggacaccg gaccaccagg gatgggcagg attttgatcc ccgtgccaag |
| 840961 | tatagttgga ccaataacgg cctccctgcg cctggtccgg ccccaccttt ctaatccaag |
| 841021 | tatccagaag agcgttgatc ccgccgttca tcctggtgtt gcccattttt ccaatgatcc |
| 841081 | caagcaccgg catcccagct cggtgcttga aacagcgggt accggccccc ttcatcatct |
| 841141 | cgatcatttc aggcgcatat ccctgctccc ggagacgcct ggccccagcc tcgccactat |
| 841201 | accgcgtggc gataatgatc atggccttgg ccgcataagt gaacgccgta tcccaagaca |
| 841261 | ctcgaagcat gtcatccagg aaccggctat cgaatttata cttgcgcttg gtttccggcg |
| 841321 | tcagttcggg agcaccatca tccatccact gcttccatcc ctttcgcatc aagggcccct |
| 841381 | tcaaccgata cggcccgtag acacgccgat ggaacgtaaa ccccttcagg cacatacgag |
| 841441 | gattgtgcgc gaacgtcccc cgatttccat aaagatcttc ataggtctgg tggtcataat |
| 841501 | tttgctcaac gcgcatgacc acgccgtttc taacaaatgc ccgcacccgg caggcatgcg |
| 841561 | tgtcattggg cgagcaaacc catgtaaatg atgaatcgta tcggtactga tcatgataga |
| 841621 | cacgctccca ggaccgatct ggatactcgc cgagcgggtt tccaacctca ataaccggtt |
| 841681 | gcagcgcggt taacgccaag actttatcgg caaccgccac agcggcaacc gtccccacgg |
| 841741 | ataccttcaa aaactgccta cgcgacaaga acat |
| SEQ ID NO: 12 | |
| 1 | mflsrrqflk vsvgtvaava vadkvlalta lqpvievgnp lgeypdrswe rvyhdqyryd |
| 61 | ssftwvcspn dthacrvraf vrngvvmrve qnydhqtyed lygnrgtfah nprmclkgft |
| 121 | fhrrvygpyr lkgplmrkgw kqwmddgape ltpetkrkyk fdsrflddml rvswdtafty |
| 181 | aakamiiiat rysgeagarr lreqgyapem iemmkgagtr cfkhragmpv lgiigkmgnt |
| 241 | rmngginall dtwirkvgpd qaqggrywsn ytwhgdqnpa hpwwsgvqgs dvdlsdmrfs |
| 301 | klntswgknf venkmpeahw kleciergar vvvitpeynp tayradywmp lrpqsdgalf |
| 361 | lgamkiivde nmhdvdflks ftdapilvrt dtlqyldprd vipdykfpdf sksysgriqs |
| 421 | lkpeqiqrlg gmmvwdlnkk qavplhreqv gwhyvnsgid aaltgtyrvk llngreidam |
| 481 | piwqmymvhf qdydldtvhq itrtpkdliv rwardsgtik paaihngegt chyfhqtina |
| 541 | rgaamvliit gnvgkfgtgq htwagnykag awtatpwsga glsvhtgedp fnitldpnah |
| 601 | gkeihtrsyy ygeevgywnh gdtalivntp kygrkvftgk thmptpskfr wvtnvnvvnn |
| 661 | akhhydmvkn vdpnieclit qdiemtsdin hadiafacns wmeftypemt itvsnpwvqi |
| 721 | wkggirplyd trndldtfag vaaklsdmtg dkrmkdyfam vyanrvdvya qrmldasstf |
| 781 | ygysadvmlk sekgwmvmvr typrhpfwee tneskpmwtr sgryenyrpe aeaieygenf |
| 841 | ishregpeat pylpnaiftt npyvrpddyg ipitaqhhdd ktvrniklsw deikrhsnpl |
| 901 | wekgyqfycv tpktrhrvhs qwsvndwvqi yesnfgdpyr mdkrtpgvge hqlhinpqaa |
| 961 | kdrgindgdy vyvdgnpvdr pyrgwkpsdp yykvarlmir akynpaypyh vtmakhapfv |
| 1021 | atpksvkghe trpdgraiai dtgyqsnfry gcqqsftrnw lmpmhqtdsl pgkhaiawkf |
| 1081 | kwgyqvdhha intvpkecli ritkaedggi gargpwepvr tgftpgqene fmikwlkgeh |
| 1141 | ikikv |
| SEQ ID NO: 13 | |
| | ttaca accaggtcac |
| 837001 | tcgctctgcc ggtctgatat aaatcggttc ttcgacttga atacgggcca cctctttgcc |
| 837061 | tgacttgtta aaaccaagaa ccgtgtcgtt gtacatctca aaccgcttgc catgaatttg |
| 837121 | ggtctcaaag acttttggcc caggaatcac gtcatagcgg aagatgatct gttgactggc |
| 837181 | tcgccacaac tgaaggacgg ccagcaattc ccggcttggg acgagatatt tttcgattgc |
| 837241 | gttgtctacg ccaggaccga acatctgtct cgcatagcct cgcgggctat gccgtggagg |
| 837301 | aatgtagaag ccgttcggtt ccgtccccca ttgcgggtag aggggtaagg ccacttgttc |
| 837361 | gacacggatg gcgtaataca gcggatgcca ccgatcctca gcccacagac cgtcttctcc |
| 837421 | gatacgaact aagctctgca ttctgatctt tcctacgcag gctgccatac accgcgtttc |
| 837481 | cattggttct ccgccggtaa gaggatcttt tccttcgatg cgcggataac aggcaataca |
| 837541 | cttttctgag accctggtgg tgcctcgata catgggcttt ttgtatgggc actgttcaac |
| 837601 | gcattttttg tatcctcgac atcgattctg atcgatgaga acaattccat cttctggccg |
| 837661 | tttgtagatc gcttttcttg gacaagcggc taggcagcca gggtaggtgc aatggttaca |
| 837721 | gatacgttgg aggtaaaaga aaaacgtttc atgctccggc aggctgctgc cggtcatttt |
| 837781 | ccagggctca tccttcgaga aacctgtctt gtcgatcccc tcgaccagcg cccgcatcga |
| 837841 | cgttgccgta tcttcgtaga tattgacaaa ccgccactcc tggtctgttg ggatgtagcc |
| 837901 | gattgcagcc tgccccactt tggcccctgc atcgaaaatg gtcatccctt cgaagacccc |
| 837961 | gtagggtgca tggtgtttcc gtcctactcg aacgttccac acctggccgc cgggattgac |
| 838021 | ctgctcgata agctgagtga ttttgacatc gtaaaattgc gggtacccgc catagggctt |
| 838081 | cgtctcgaca ttgttccacc acatgtactc ctgacctttc gagaaaagcc aggttgactt |
| 838141 | atccgccatg gaacacgtct gacaggccag acatcgattg atgttaaaca caaaggcaaa |
| 838201 | ttgccatttg ggatgccgct cctcataggg ataaagcatc tttcgtccta actgccagtt |
| 838261 | ataaacttca ggcat |
| SEQ ID NO: 14 | |
| 1 | mpevynwqlg rkmlypyeer hpkwqfafvf ninrclacqt csmadkstwl fskgqeymww |
| 61 | nnvetkpygg ypqfydvkit qlieqvnpgg qvwnvrvgrk hhapygvfeg mtifdagakv |
| 121 | gqaaigyipt dqewrfvniy edtatsmral vegidktgfs kdepwkmtgs slpehetfff |
| 181 | ylqricnhct ypgclaacpr kaiykrpedg ivlidqnrcr gykkcveqcp ykkpmyrgtt |
| 241 | rvsekciacy priegkdplt ggepmetrcm aacvgkirmq slvrigedgl waedrwhply |
| 301 | yairveqval plypqwgtep ngfyipprhs prgyarqmfg pgvdnaieky lvpsrellav |
| 361 | lqlwrasqqi ifrydvipgp kvfetqihgk rfemyndtvl gfnksgkeva riqveepiyi |
| 421 | rpaervtwl |
| SEQ ID NO: 15 | |
| 1 | atgaaaacac tatggcaaaa taatccgtgt gcaacaatgg ccaaaaccat cagttaccgg |
| 61 | aatgctaacg cactaaagca gccctttacg aaaggcttgt tattcctggg tacgctactt |
| 121 | tcggtgtata tgttgaccct acagcctgtc atggcgcacg gggaaaagaa cctggaaccc |
| 181 | tatgtcagaa tgcgtaccgt ccaatggtat gacgtgcaat ggtccaagca gaaatttaat |
| 241 | gtcaacgatg aaattagcgt aaccggtaaa tttcatgtgg ccgaagattg gccgatcagc |
| 301 | gtacccaagc cggatgcggc gttcttaaat atctcaacac caggccccgt gctgatcaga |
| 361 | accgaacgtt acttaaacgg caagccctac atgaattcag tggccttaca accaggcggc |
| 421 | gactatgact tcaaggttgt cctgaaagga cgcttaccag gacgttacca catccatcct |
| 481 | ttctttaacc taaaggatgc agggcaagtc atggggccgg gcgcatggtt ggatattgca |
| 541 | ggcgatgcca gcgattttac caataacgtc cagaccatca atggcgaact ggtcgatatg |
| 601 | gaaaacttcg ggttgggtaa cggcatcttc tggcacagct tttgggcttt gttgggtacg |
| 661 | gcctggctgc tttggtgggt acgccgcccc ttgtttattg agcgttaccg gatgttgcaa |
| 721 | gcaggcttgg aagatgaatt ggtgactcca ttggacagaa atattggcaa agcaatagtc |
| 781 | atcggcgtgc ctgttctggt gtttatgttt tataccatga cggtgaacaa atatcccaag |
| 841 | gccatacctt tacaagcctc actagaccaa atcctgcctc tttctgccca agtcaatgcc |
| 901 | ggcgtagtcg atgtcgaaac ggtgcggaca gaataccgcg tcccaaaaag atcgatgact |
| 961 | gtcagcttaa agatcaaaaa tggcagcgac aagccgattc agataggtga atttgcaacg |
| 1021 | ggcggtgtac gcttccttaa ccaagctgta tctgtacctg accagaacaa tgcagaaagt |
| 1081 | gttatcgcga aagaaggctt aatattggat aatccagccc ccatccagcc aggtgaacaa |
| 1141 | cgtacagtgt taatgaccgc aagcgatgcc ttgtgggagt cagaaaaact ggacggcctg |
| 1201 | attaacgatg ccgacagccg tattggcggc ttgatctttt tcttcgacag tgagggtgaa |
| 1261 | cgcactattt ccagcatcac ctcggctgtc attcctaaat ttgattaa |
| SEQ ID NO: 16 | |
| 1 | mktlwqnnpc atmaktisyr nanalkqpft kgllflgtll svymltlqpv mahgeknlep |
| 61 | yvrmrtvqwy dvqwskqkfn vndeisvtgk fhvaedwpis vpkpdaafln istpgpvlir |
| 121 | terylngkpy mnsvalqpgg dydfkvvlkg rlpgryhihp ffnlkdagqv mgpgawldia |
| 181 | gdasdftnnv qtingelvdm enfglgngif whsfwallgt awllwwvrrp lfieryrmlq |
| 241 | agledelvtp ldrnigkaiv igvpvlvfmf ytmtvnkypk aiplqasldq ilplsaqvna |
| 301 | gvvdvetvrt eyrvpkrsmt vslkikngsd kpiqigefat ggvrflnqav svpdqnnaes |
| 361 | viakeglild npapiqpgeq rtvlmtasda lwesekldgl indadsrigg lifffdsege |
| 421 | rtissitsav ipkfd |
| SEQ ID NO: 17 | |
| 1 | atgtcagcaa aactttcaaa gccaacgttt aagccgtata ccggcgagaa ggcgcgtatc |
| 61 | acccgcgctt acgactacct gatcctagta ttggcgctgt tcttgttcat cggttctttc |
| 121 | catctgcatt ttgccctcac tgtgggcgac tgggattttt gggtagactg gaaggacagg |
| 181 | caatggtggc cattggtcac cccactcatt ggcattacct ttccggcggc agtacaggcc |
| 241 | gtactatgga gtaacttccg cttgccattg ggtgcaaccc tgtgtgttgc ctgtttgtcg |
| 301 | ataggtacct ggattgcccg tgtctttgca taccactact ggaattattt tcccatcaac |
| 361 | atggtgatgc catcgacact gctgcctagt gcgctggtct tggacggcat cctcatgtta |
| 421 | agtaatagcc tgacagtgac cgctattttc ggcggctctg ctttcgcctt actgttctac |
| 481 | cctgcaaact ggcccatctt cggtatgttc catctccccg ttgaagcggg caacagccaa |
| 541 | ttgaccctgg ccgatatgtt tggcttccag tacatccgta ccggtatgcc ggaatatctt |
| 601 | cgtattattg agcgggggac gttacgtact tatggccaaa ttgccacacc gctgtcggcc |
| 661 | ttttgctcag cgctgttatg cactttgatg tacaccttgt ggtggcatat cggcaaatgg |
| 721 | tttgccacga cccgttatct taaaagaatc taa |
| SEQ ID NO: 18 | |
| 1 | msaklskptf kpytgekari traydylilv lalflfigsf hlhfaltvgd wdfwvdwkdr |
| 61 | qwwplvtpli gitfpaavqa vlwsnfrlpl gatlcvacls igtwiarvfa yhywnyfpin |
| 121 | mvmpstllps alvldgilml snsltvtaif ggsafallfy panwpifgmf hlpveagnsq |
| 181 | ltladmfgfq yirtgmpeyl riiergtlrt ygqiatplsa fcsallctlm ytlwwhigkw |
| 241 | fattrylkri |
| SEQ ID NO: 19 | |
| 1 | atggctacaa ccactgaaaa aatcaaggta ataaccgaac aggccaaaat gccaccctgg |
| 61 | tatttgaagg atttataccg ctatctgtcg gctttcggca tactgaccgc catctatatg |
| 121 | ggtttccgta tttatcaggg ggcgtatggt gtctcaacag gattggattc aaccgccccc |
| 181 | gattttgatg tctactggat gcgtctgttc aactttaacg tgacttttgt tacgcttttt |
| 241 | gcaggcgttt catggggatg gttatggttt acccgggata aaaacctgga caagcttgaa |
| 301 | cctaaggaag aaatccgccg ctattttacg ttgaccatgt tcattagcgt ctataccttt |
| 361 | gctgtatatt gggctggcag ttactttgcc gagcaagata actcctggca tcaggtcgct |
| 421 | attcgagaca caccttttac cgccaaccat atcattgaat tttatttcaa tttccccatg |
| 481 | tacattatcc ttggcggttg cgcctggctt tatgccagaa cacggctgcc gctttatgcc |
| 541 | aaaggcattt cactgccgtt gacgctggct gttgtcgggc cttttatgat attggtgagt |
| 601 | gtcggtttta atgaatgggg gcataccttc tggtttcgtg aggagttttt tgctgcgccg |
| 661 | atccattacg gcttcgtgat tggggtttgg tttgcgcatg gcgtgggggg tatattgctg |
| 721 | caaggtgtga cccgtttgat tgagttgcta gacgcacagg aagacgtggc ttaa |
| SEQ ID NO: 20 | |
| 1 | matttekikv iteqakmppw ylkdlyryls afgiltaiym gfriyqgayg vstgldstap |
| 61 | dfdvywmrlf nfnvtfvtlf agvswgwlwf trdknldkle pkeeirryft ltmfisvytf |
| 121 | avywagsyfa eqdnswhqva irdtpftanh iiefyfnfpm yiilggcawl yartrlplya |
| 181 | kgislpltla vvgpfmilvs vgfnewghtf wfreeffaap ihygfvigvw fahgvggill |
| 241 | qgvtrliell daqedva |

1. A bacterial composition comprising at least one species of bacteria wherein the at least one species of bacteria is capable of catalyzing complete nitrification.

2. The bacterial composition of claim 1, wherein the at least one species of bacteria is selected from Nitrospira, Nitrosomonas, Nitrosococcus, Nitrosospira, Nitrobacter, Nitrospina, Nitrococcus, and combinations of thereof.

3. The bacterial composition of claim 1, further comprising at least two species of nitrifying bacteria.

4. The bacterial composition of claim 1, wherein the composition is in a form selected from the group consisting of liquid, concentrated, frozen, freeze-dried, and powdered.

5. The bacterial composition of claim 1, further comprising at least one additional species of bacteria which serves metabolic functions ancillary to the nitrifying bacteria.

6. The bacterial composition of claim 1, wherein the composition comprises at least one species of Nitrospira bacteria.

7. The bacterial composition of claim 1, further comprising at least one urease inhibitor.

8. The bacterial composition of claim 1, wherein the at least one species of bacteria capable of catalyzing complete nitrification is a recombinant host comprising at least one nucleic acid encoding a polypeptide capable of oxidizing ammonia or ammonium to nitrite and at least one nucleic acid encoding a polypeptide capable of oxidizing nitrite to nitrate, wherein:
at least one nucleic acid encoding a polypeptide capable of oxidizing ammonia or ammonium to nitrite is recombinant;
at least one nucleic acid encoding a polypeptide capable of oxidizing nitrite to nitrate is recombinant; or
at least one nucleic acid encoding a polypeptide capable of oxidizing ammonia or ammonium to nitrite is recombinant and at least one nucleic acid encoding a polypeptide capable of oxidizing nitrite to nitrate is recombinant.

9. The bacterial composition of claim 8, further wherein the at least one polypeptide capable of oxidizing ammonia or ammonium to nitrite is selected from ammonia monooxygenase, hydroxylamine oxidoreductase, hydroxylamine dehydrogenase, methane monooxygenase, and combinations thereof.

10. The bacterial composition of claim 8, further wherein the at least one polypeptide capable of oxidizing ammonia or ammonium to nitrite is 90% homologous to SEQ ID NO: 2, 4, 6, 8, 10, 16, 18, 20, or a combination of these sequences.

11. The bacterial composition of claim 8, further wherein the at least one polypeptide capable of oxidizing nitrite to nitrate is nitrite oxidoreductase.

12. The bacterial composition of claim 8, further wherein the at least one polypeptide capable of oxidizing nitrite to nitrate is 90% homologous to SEQ ID NO: 12, 14, or a combination of these sequences.

13. A method of treating a fabric, the method comprising applying to the fabric, an effective amount of the bacterial composition of any of the previous claims.

14. The method of claim 13, wherein the applying an effective amount of the bacterial composition comprises spraying the bacterial composition on to the fabric at least once.

15. The method of claim 13, wherein prior to the application of the effective amount of bacterial composition, the fabric is wiped with an applicator dampened with water.

16. The method of claim 13, wherein the fabric is an article of clothing.

17. The method of claim 13, wherein the fabric is denim.

18. A kit useful for the treatment of a fabric, the kit comprising an effective amount of the bacterial composition of claim 1, and instructions for use.

19. The kit of claim 18, further comprising an applicator used for applying the bacterial composition to a fabric.

20. The kit of claim 18, wherein the bacterial composition is packaged as a concentrate which can be diluted with water.
